SEQUENCE LISTING (26 sequences)

SEQ ID NO: 1 - DNA, 60 bp, synthetic construct
  source 1..60: mol_type = other DNA
atggagacagacacactcctgctatgggtactgctgctctgggttccaggttccactggc

SEQ ID NO: 2 - protein, 23 aa, synthetic construct
METDTLLLWVLLLWVPGSTGAAS

SEQ ID NO: 3 - DNA, 711 bp, Homo sapiens
  source 1..711: mol_type = other DNA
atggagacagacacactcctgctatgggtactgctgctctgggttccaggttccactggcgccgctagcgaaattgtgctgacccagagcccggcgaccctgagcctgagcccgggcgaacgcgcgaccctgagctgcagcgcgagcagcagcattagctatatgcattggtatcagcagaaaccgggccaggcgccgcgcctgctgatttatgataccagcaaactggcgaccggcattccggcgcgctttagcggcagcggcagcggcaccgattttaccctgaccattagcagcctggaaccggaagattttgcggtgtattattgccatcagcgcagcagctatccgtatacctttggcggcggcaccaaactggaaattaaacgtacagtagctgctccgtccgtctttattttcccgccgtctgacgaacagctcaaaagcgggactgcatcagttgtctgtctcctcaacaatttttacccgcgagaggctaaggttcaatggaaagttgataacgccctccagagtggaaactctcaggagagtgtaactgagcaagattccaaagattcaacctatagtctttcaagtaccttgactctttctaaagcggattatgagaaacataaagtgtatgcctgcgaagtgacccatcaggggctttcatcacccgtgacgaagtcctttaaccgaggcgaatgctaa

SEQ ID NO: 4 - protein, 106 aa, Homo sapiens
EIVLTQSPATLSLSPGERATLSCSASSSISYMHWYQQKPGQAPRLLIYDTSKLATGIPARFSGSGSGTDFTLTISSLEPEDFAVYYCHQRSSYPYTFGGGTKLEIK

SEQ ID NO: 5 - protein, 213 aa, Homo sapiens
EIVLTQSPATLSLSPGERATLSCSASSSISYMHWYQQKPGQAPRLLIYDTSKLATGIPARFSGSGSGTDFTLTISSLEPEDFAVYYCHQRSSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - DNA, 720 bp, Homo sapiens
  source 1..720: mol_type = other DNA
atggagacagacacactcctgctatgggtactgctgctctgggttccaggttccactggcgccgctagccaggccgtagtgacacaggaaccgtctttgacggtgtctccgggaggtaccgtcaccttgacgtgtgggtccagcactggagctgtaacaacgagcaattacgcgaattgggtgcaggagaagccaggtcaggcttttaggggtcttatcggagggactaataaaagggctccaggcacgccggcaagattctcagggtccctgctgggggggaaagcggcactcaccctttctggtgctcagccagaggatgaggccgaatattattgtgccttgtggtattctaatttgtgggtctttggaggcgggacaaaactcactgtattgtcatctgcgtcaacgaagggaccttctgtattccccttggcaccatccagtaaatctaccagtgggggtaccgctgccctcggttgccttgtaaaagattactttccggagcccgtcaccgtgtcctggaacagcggggcattgaccagtggtgtccacacttttcccgcagtactccaaagctccggcctctacagtctctcttcagttgtgacggttcctagctcttcccttggtacgcagacttatatctgcaacgtcaaccacaaaccttccaatactaaggtagacaaaaaggtggagcccaaatcttgttaaatgcat

SEQ ID NO: 7 - protein, 109 aa, Homo sapiens
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 8 - protein, 214 aa, Homo sapiens
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 9 - DNA, 2133 bp, Homo sapiens
  source 1..2133: mol_type = other DNA
atggagacagacacactcctgctatgggtactgctgctctgggttccaggttccactggcgccgctagccaggtgcagctggtgcagagcggcagcgaactgaaaaaaccgggcgcgagcgtgaaagtgagctgcaaagcgagcggctatacctttaccaactatggcatgaactgggtgcgccaggcgccgggccagggcctggaatggatgggctggattaacacctataccggcgaaccgacctatgcggatgattttaaaggccgctttgtgtttagcctggataccagcgtgagcaccgcgtatctgcagattagcagcctgaaagcggaagataccgcggtgtattattgcgcgcgctggctgcgcgattttgattattggggcgcgggcaccaccgtgaccgtgagcagcacaaaaggtccttccgtatttccgctcgcacccagctctaagtcaacctctggcggtactgcagccctgggttgcctcgtaaaggactattttcctgagccagtaacagtttcttggaacagcggggcacttacgagcggtgttcatacgttccctgcagtgttgcaatccagcggcctttattcattgtcttcagttgtaacggttccttctagtagtttggggacccagacatatatctgcaacgtgaaccataagccaagcaataccaaagttgataagaaggtcgaacctaagtcctgcgacggcgggggaggatctggcgggggaggcagtgaggtacaattgttggagtcaggcggcggtcttgtccaaccgggagggtccctgagactgtcctgtgcggccagcgggtttactttttcaacatatgccatgaactgggttcggcaagcaccaggtaagggactggaatgggttagtcgaattaggtccaagtataataattacgcaacctactacgctgactctgtcaaggggcggtttaccatatctagggatgactccaaaaacacattgtacttgcaaatgaacagcctgagggcagaagacaccgcagtctactattgtgtacgccacggaaacttcgggaatagttatgtctcctggttcgcttactggggtcagggaacactggtcacagtctcatcagccagtgtagcggccccgtccgttttcatattccctccttccgacgagcagttgaaaagcggtacggcgagcgttgtgtgcttgttgaacaacttctacccacgcgaagccaaggtccaatggaaggtagacaacgcactgcagagtggtaactcacaggaatcagtgacggaacaggactcaaaagatagtacttacagtctttcttccacactgacactcagtaaggccgattatgagaaacataaagtatacgcatgtgaagtaactcaccagggtctcagttcaccagtaactaagtctttcaatcgcggggaatgcgacaaaacacacacctgtcccccctgtccagccccagaggcagctggcggccctagtgtgttcttgttcccgcccaagccaaaagatacactgatgattagccggacccctgaggtaacttgtgtggtggtggacgtgtctcatgaggacccagaggtaaaattcaactggtacgtagacggcgtcgaggtccataatgccaaaaccaagccacgggaggagcagtataattccacttatcgcgtagtctctgtacttacagttcttcaccaagattggttgaacggaaaagaatacaagtgtaaagttagcaataaggcgctcggagctccgatcgaaaaaacaatctccaaagcaaaagggcaaccccgagaaccacaggtatacaccctgccgccgtgccgagacgagctgacgaaaaaccaagtgtccctgtggtgcttggtgaagggcttttatccaagtgacattgcagttgaatgggagtctaacggacagcctgaaaataactataagaccacgccaccagtccttgatagcgatggatctttttttctctatagcaagttgactgtagataaatcacgatggcaacaaggcaatgtcttttcatgcagcgttatgcatgaggctctgcacaaccactacacgcagaagagcctctccctgtctcccgggaaatga

SEQ ID NO: 10 - protein, 116 aa, Homo sapiens
QVQLVQSGSELKKPGASVKVSCKASGYTFTNYGMNWVRQAPGQGLEWMGWINTYTGEPTYADDFKGRFVFSLDTSVSTAYLQISSLKAEDTAVYYCARWLRDFDYWGAGTTVTVSS

SEQ ID NO: 11 - protein, 125 aa, Homo sapiens
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 12 - protein, 687 aa, Homo sapiens
QVQLVQSGSELKKPGASVKVSCKASGYTFTNYGMNWVRQAPGQGLEWMGWINTYTGEPTYADDFKGRFVFSLDTSVSTAYLQISSLKAEDTAVYYCARWLRDFDYWGAGTTVTVSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1404 bp, Homo sapiens
  source 1..1404: mol_type = other DNA
atggagacagacacactcctgctatgggtactgctgctctgggttccaggttccactggcgccgctagccaggtgcagctggtgcagagcggcagcgaactgaaaaaaccgggcgcgagcgtgaaagtgagctgcaaagcgagcggctatacctttaccaactatggcatgaactgggtgcgccaggcgccgggccagggcctggaatggatgggctggattaacacctataccggcgaaccgacctatgcggatgattttaaaggccgctttgtgtttagcctggataccagcgtgagcaccgcgtatctgcagattagcagcctgaaagcggaagataccgcggtgtattattgcgcgcgctggctgcgcgattttgattattggggcgcgggcaccaccgtgaccgtgagcagcaccaaaggtccaagcgtatttccactcgcaccgtcctccaaatcaacgagtggaggtactgcggcgttgggatgcttggtgaaagactatttcccagagccggtgacagttagttggaactcaggcgcgctgactagtggtgttcacacatttcctgcagttttgcaatcctcaggcctctatagcctgtcaagcgtcgtgacagtacctagcagctcacttggcacgcagacatatatttgtaatgtcaaccataagccctctaacacaaaggtagataagaaggttgaacccaagtcctgtgacaaaacgcacacttgtccaccatgtccagcgcccgaagcagccgggggcccaagcgtgttcctcttccctcccaagccaaaagacacccttatgatctcaaggactccagaagtgacatgcgtagtcgttgacgtaagtcacgaggatccggaagtgaagttcaactggtacgtggacggtgtggaggtacataacgcgaagactaagcccagagaagaacaatataactcaacctaccgggtcgtttctgtgctcacagtgctccaccaggactggcttaacggaaaagagtataaatgcaaagtatctaataaagcgctcggagcgcccatagagaaaactatttctaaagcaaaaggtcaaccacgggagccgcaggtttgcacacttccaccgtccagggatgaacttactaagaaccaggtatctctttcttgtgccgtgaaaggtttttatcctagtgacatcgctgtcgagtgggagagcaacggtcagccggagaataactataagaccacacctccggttctggattctgacggctctttcttcctggtatctaagcttacagtcgataaaagtcgatggcaacaagggaatgtttttagctgctctgtgatgcatgaggctctgcacaaccactacacgcagaagagcctctccctgtctcccgggaaatga

SEQ ID NO: 14 - protein, 444 aa, Homo sapiens
QVQLVQSGSELKKPGASVKVSCKASGYTFTNYGMNWVRQAPGQGLEWMGWINTYTGEPTYADDFKGRFVFSLDTSVSTAYLQISSLKAEDTAVYYCARWLRDFDYWGAGTTVTVSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 15 - DNA, 1596 bp, Homo sapiens
  source 1..1596: mol_type = other DNA
atggagacagacacactcctgctatgggtactgctgctctgggttccaggttccactggcgccgctagccaggtgcaactcgtacaaagcggttccgaactgaagaaacctggtgcgagcgtaaaggtgtcctgtaaagcatctggatacaccttcaccaactacggaatgaactgggttcgacaggcaccgggccaaggcttggagtggatggggtggatcaacacctacacgggtgaacctacgtacgcagatgatttcaaaggtcgcttcgtgttttccctggatacttctgtgtccacagcgtacctccagatttccagtctgaaggccgaggacacagccgtatattactgtgctcggtggttgcgcgattttgattattggggcgcaggtactactgtgacagtaagctccggcggaggcgggtcagggggcggtggatccggcggagggggatcagaaatcgtgctcactcaatcaccagcgaccttgagcttgtcacctggggagcgggcgacgctgtcctgtagtgcgtcatcatctatttcatatatgcactggtatcaacagaagccaggacaggcgcctcgcctgttgatttacgatactagcaaattggcaacggggatacccgctcgattcagcggcagcggatctgggacggatttcacattgaccatttctagccttgaaccagaggacttcgcagtatattactgccatcagaggtccagctatccatacacatttggcggagggacgaaattggaaatcaaggggggaggcgggtctgggggaggcggaagtgggggaggaggaagcgaagttcagctgctcgaatccggcggcggccttgttcagccaggtggtagcttgaggctcagttgtgctgcatctgggtttacattctcaacttatgcgatgaactgggtgaggcaagcacctggaaagggacttgagtgggtctcaagaattcgctccaaatacaacaactatgcgacgtattacgcagactcagtgaaaggacggtttacgatatcacgggacgattcaaagaatacactgtatttgcagatgaattctcttagggccgaagacactgccgtatactattgtgtacgccacggtaattttggcaatagctatgtatcttggttcgcgtactggggccaaggcacccttgttactgtgtctagtgggggcggggggagtggtggcggaggaagtggcggggggggatctcaagcggtggttactcaagagccctcccttactgtttctccgggcgggacggtcaccttgacttgtggcagttcaacaggggcagtcactactagtaattatgcgaattgggtccaagaaaagccgggccaagctttccggggactcatcggaggaacaaataaaagggcacccggcacacccgcgcgcttttccgggagtcttctgggcggcaaggcagccctcactctctctggggctcaacctgaggacgaggctgagtactattgtgccctctggtactcaaacctgtgggtctttggagggggaaccaagcttacggtcttgcatcatcaccatcatcattaatga

SEQ ID NO: 16 - protein, 530 aa, Homo sapiens
METDTLLLWVLLLWVPGSTGAASQVQLVQSGSELKKPGASVKVSCKASGYTFTNYGMNWVRQAPGQGLEWMGWINTYTGEPTYADDFKGRFVFSLDTSVSTAYLQISSLKAEDTAVYYCARWLRDFDYWGAGTTVTVSSGGGGSGGGGSGGGGSEIVLTQSPATLSLSPGERATLSCSASSSISYMHWYQQKPGQAPRLLIYDTSKLATGIPARFSGSGSGTDFTLTISSLEPEDFAVYYCHQRSSYPYTFGGGTKLEIKGGGGSGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSGGGGSGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLHHHHHH

SEQ ID NO: 17 - DNA, 2277 bp, Homo sapiens
  source 1..2277: mol_type = other DNA
atggagacagacacactcctgctatgggtactgctgctctgggttccaggttccactggcgccgctagccaggtgcaactcgtacaaagcggttccgaactgaagaaacctggtgcgagcgtaaaggtgtcctgtaaagcatctggatacaccttcaccaactacggaatgaactgggttcgacaggcaccgggccaaggcttggagtggatggggtggatcaacacctacacgggtgaacctacgtacgcagatgatttcaaaggtcgcttcgtgttttccctggatacttctgtgtccacagcgtacctccagatttccagtctgaaggccgaggacacagccgtatattactgtgctcggtggttgcgcgattttgattattggggcgcaggtactactgtgacagtaagctccggcggaggcgggtcagggggcggtggatccggcggagggggatcagaaatcgtgctcactcaatcaccagcgaccttgagcttgtcacctggggagcgggcgacgctgtcctgtagtgcgtcatcatctatttcatatatgcactggtatcaacagaagccaggacaggcgcctcgcctgttgatttacgatactagcaaattggcaacggggatacccgctcgattcagcggcagcggatctgggacggatttcacattgaccatttctagccttgaaccagaggacttcgcagtatattactgccatcagaggtccagctatccatacacatttggcggagggacgaaattggaaatcaaggggggaggcgggtctgggggaggcggaagtgggggaggaggaagcgaagttcagctgctcgaatccggcggcggccttgttcagccaggtggtagcttgaggctcagttgtgctgcatctgggtttacattctcaacttatgcgatgaactgggtgaggcaagcacctggaaagggacttgagtgggtctcaagaattcgctccaaatacaacaactatgcgacgtattacgcagactcagtgaaaggacggtttacgatatcacgggacgattcaaagaatacactgtatttgcagatgaattctcttagggccgaagacactgccgtatactattgtgtacgccacggtaattttggcaatagctatgtatcttggttcgcgtactggggccaaggcacccttgttactgtgtctagtgggggcggggggagtggtggcggaggaagtggcggggggggatctcaagcggtggttactcaagagccctcccttactgtttctccgggcgggacggtcaccttgacttgtggcagttcaacaggggcagtcactactagtaattatgcgaattgggtccaagaaaagccgggccaagctttccggggactcatcggaggaacaaataaaagggcacccggcacacccgcgcgcttttccgggagtcttctgggcggcaaggcagccctcactctctctggggctcaacctgaggacgaggctgagtactattgtgccctctggtactcaaacctgtgggtctttggagggggaaccaagcttacggtcttgtctagagaaaacctgtattttcagggcactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 18 - protein, 22 aa, synthetic construct
ETDTLLLWVLLLWVPGSTGAAS

SEQ ID NO: 19 - protein, 735 aa, Homo sapiens
QVQLVQSGSELKKPGASVKVSCKASGYTFTNYGMNWVRQAPGQGLEWMGWINTYTGEPTYADDFKGRFVFSLDTSVSTAYLQISSLKAEDTAVYYCARWLRDFDYWGAGTTVTVSSGGGGSGGGGSGGGGSEIVLTQSPATLSLSPGERATLSCSASSSISYMHWYQQKPGQAPRLLIYDTSKLATGIPARFSGSGSGTDFTLTISSLEPEDFAVYYCHQRSSYPYTFGGGTKLEIKGGGGSGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSGGGGSGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSRENLYFQGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 18 bp, synthetic construct
  source 1..18: mol_type = other DNA
atttaggtgacactatag

SEQ ID NO: 22 - DNA, 5720 bp, Homo sapiens
  source 1..5720: mol_type = other DNA
atttaggtgacactatagaatacaagcttacatttgcttctgacacaactgtgttcactagcaacctcaaacagacaccatggagacagacacactcctgctatgggtactgctgctctgggttccaggttccactggcgccgctagccaggtgcaactcgtacaaagcggttccgaactgaagaaacctggtgcgagcgtaaaggtgtcctgtaaagcatctggatacaccttcaccaactacggaatgaactgggttcgacaggcaccgggccaaggcttggagtggatggggtggatcaacacctacacgggtgaacctacgtacgcagatgatttcaaaggtcgcttcgtgttttccctggatacttctgtgtccacagcgtacctccagatttccagtctgaaggccgaggacacagccgtatattactgtgctcggtggttgcgcgattttgattattggggcgcaggtactactgtgacagtaagctccggcggaggcgggtcagggggcggtggatccggcggagggggatcagaaatcgtgctcactcaatcaccagcgaccttgagcttgtcacctggggagcgggcgacgctgtcctgtagtgcgtcatcatctatttcatatatgcactggtatcaacagaagccaggacaggcgcctcgcctgttgatttacgatactagcaaattggcaacggggatacccgctcgattcagcggcagcggatctgggacggatttcacattgaccatttctagccttgaaccagaggacttcgcagtatattactgccatcagaggtccagctatccatacacatttggcggagggacgaaattggaaatcaaggggggaggcgggtctgggggaggcggaagtgggggaggaggaagcgaagttcagctgctcgaatccggcggcggccttgttcagccaggtggtagcttgaggctcagttgtgctgcatctgggtttacattctcaacttatgcgatgaactgggtgaggcaagcacctggaaagggacttgagtgggtctcaagaattcgctccaaatacaacaactatgcgacgtattacgcagactcagtgaaaggacggtttacgatatcacgggacgattcaaagaatacactgtatttgcagatgaattctcttagggccgaagacactgccgtatactattgtgtacgccacggtaattttggcaatagctatgtatcttggttcgcgtactggggccaaggcacccttgttactgtgtctagtgggggcggggggagtggtggcggaggaagtggcggggggggatctcaagcggtggttactcaagagccctcccttactgtttctccgggcgggacggtcaccttgacttgtggcagttcaacaggggcagtcactactagtaattatgcgaattgggtccaagaaaagccgggccaagctttccggggactcatcggaggaacaaataaaagggcacccggcacacccgcgcgcttttccgggagtcttctgggcggcaaggcagccctcactctctctggggctcaacctgaggacgaggctgagtactattgtgccctctggtactcaaacctgtgggtctttggagggggaaccaagcttacggtcttgtctagagaaaacctgtattttcagggcactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatgagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcagtcgactctagaaaaaaaaaaaaaaaaaaaaaaaaaaaaaaaaaaaaaaaaaggatccccgggcgagctcccaaaaaaaaaaaaaaaaaaaaaaaaaaaaaaccgaattcctgcagctcgagaaaaaaaaaaaaaaaaaaaaaaaaaaaaaaaaaaaaaaaaagatctggcgcgccgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacg

SEQ ID NO: 23 - DNA, 5726 bp, Homo sapiens
  source 1..5726: mol_type = other DNA
taatacgactcactataaggagagaatacaagcttacatttgcttctgacacaactgtgttcactagcaacctcaaacagacaccatggagacagacacactcctgctatgggtactgctgctctgggttccaggttccactggcgccgctagccaggtgcaactcgtacaaagcggttccgaactgaagaaacctggtgcgagcgtaaaggtgtcctgtaaagcatctggatacaccttcaccaactacggaatgaactgggttcgacaggcaccgggccaaggcttggagtggatggggtggatcaacacctacacgggtgaacctacgtacgcagatgatttcaaaggtcgcttcgtgttttccctggatacttctgtgtccacagcgtacctccagatttccagtctgaaggccgaggacacagccgtatattactgtgctcggtggttgcgcgattttgattattggggcgcaggtactactgtgacagtaagctccggcggaggcgggtcagggggcggtggatccggcggagggggatcagaaatcgtgctcactcaatcaccagcgaccttgagcttgtcacctggggagcgggcgacgctgtcctgtagtgcgtcatcatctatttcatatatgcactggtatcaacagaagccaggacaggcgcctcgcctgttgatttacgatactagcaaattggcaacggggatacccgctcgattcagcggcagcggatctgggacggatttcacattgaccatttctagccttgaaccagaggacttcgcagtatattactgccatcagaggtccagctatccatacacatttggcggagggacgaaattggaaatcaaggggggaggcgggtctgggggaggcggaagtgggggaggaggaagcgaagttcagctgctcgaatccggcggcggccttgttcagccaggtggtagcttgaggctcagttgtgctgcatctgggtttacattctcaacttatgcgatgaactgggtgaggcaagcacctggaaagggacttgagtgggtctcaagaattcgctccaaatacaacaactatgcgacgtattacgcagactcagtgaaaggacggtttacgatatcacgggacgattcaaagaatacactgtatttgcagatgaattctcttagggccgaagacactgccgtatactattgtgtacgccacggtaattttggcaatagctatgtatcttggttcgcgtactggggccaaggcacccttgttactgtgtctagtgggggcggggggagtggtggcggaggaagtggcggggggggatctcaagcggtggttactcaagagccctcccttactgtttctccgggcgggacggtcaccttgacttgtggcagttcaacaggggcagtcactactagtaattatgcgaattgggtccaagaaaagccgggccaagctttccggggactcatcggaggaacaaataaaagggcacccggcacacccgcgcgcttttccgggagtcttctgggcggcaaggcagccctcactctctctggggctcaacctgaggacgaggctgagtactattgtgccctctggtactcaaacctgtgggtctttggagggggaaccaagcttacggtcttgtctagagaaaacctgtattttcagggcactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatgagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcagtcgactctagaaaaaaaaaaaaaaaaaaaaaaaaaaaaaaaaaaaaaaaaaggatccccgggcgagctcccaaaaaaaaaaaaaaaaaaaaaaaaaaaaaaccgaattcctgcagctcgagaaaaaaaaaaaaaaaaaaaaaaaaaaaaaaaaaaaaaaaaagatctggcgcgccgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacg

SEQ ID NO: 24 - DNA, 5720 bp, Homo sapiens
  source 1..5720: mol_type = other DNA
taatacgactcactataaggagaaagcttacatttgcttctgacacaactgtgttcactagcaacctcaaacagacaccatggagacagacacactcctgctatgggtactgctgctctgggttccaggttccactggcgccgctagccaggtgcaactcgtacaaagcggttccgaactgaagaaacctggtgcgagcgtaaaggtgtcctgtaaagcatctggatacaccttcaccaactacggaatgaactgggttcgacaggcaccgggccaaggcttggagtggatggggtggatcaacacctacacgggtgaacctacgtacgcagatgatttcaaaggtcgcttcgtgttttccctggatacttctgtgtccacagcgtacctccagatttccagtctgaaggccgaggacacagccgtatattactgtgctcggtggttgcgcgattttgattattggggcgcaggtactactgtgacagtaagctccggcggaggcgggtcagggggcggtggatccggcggagggggatcagaaatcgtgctcactcaatcaccagcgaccttgagcttgtcacctggggagcgggcgacgctgtcctgtagtgcgtcatcatctatttcatatatgcactggtatcaacagaagccaggacaggcgcctcgcctgttgatttacgatactagcaaattggcaacggggatacccgctcgattcagcggcagcggatctgggacggatttcacattgaccatttctagccttgaaccagaggacttcgcagtatattactgccatcagaggtccagctatccatacacatttggcggagggacgaaattggaaatcaaggggggaggcgggtctgggggaggcggaagtgggggaggaggaagcgaagttcagctgctcgaatccggcggcggccttgttcagccaggtggtagcttgaggctcagttgtgctgcatctgggtttacattctcaacttatgcgatgaactgggtgaggcaagcacctggaaagggacttgagtgggtctcaagaattcgctccaaatacaacaactatgcgacgtattacgcagactcagtgaaaggacggtttacgatatcacgggacgattcaaagaatacactgtatttgcagatgaattctcttagggccgaagacactgccgtatactattgtgtacgccacggtaattttggcaatagctatgtatcttggttcgcgtactggggccaaggcacccttgttactgtgtctagtgggggcggggggagtggtggcggaggaagtggcggggggggatctcaagcggtggttactcaagagccctcccttactgtttctccgggcgggacggtcaccttgacttgtggcagttcaacaggggcagtcactactagtaattatgcgaattgggtccaagaaaagccgggccaagctttccggggactcatcggaggaacaaataaaagggcacccggcacacccgcgcgcttttccgggagtcttctgggcggcaaggcagccctcactctctctggggctcaacctgaggacgaggctgagtactattgtgccctctggtactcaaacctgtgggtctttggagggggaaccaagcttacggtcttgtctagagaaaacctgtattttcagggcacccacacgtgccccccttgcccagcacccgaagccgcaggtggcccatcagtgtttctttttcctccaaaaccaaaagacacactcatgatctcccggacgcctgaggtgacctgtgtagtcgtagacgtatcccatgaggaccctgaagtaaagtttaactggtatgtagacggtgtggaagtacacaatgccaagactaaaccaagagaggaacagtataacagcacctatagggtagtttccgtgctcaccgttctccaccaagattggcttaacggtaaagaatataaatgtaaggtgtcaaataaggcactcggagccccgatcgaaaagaccatctctaaagcaaaaggacagcccagggagccacaagtctacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaataagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcagtcgactctagaaaaaaaaaaaaaaaaaaaaaaaaaaaaaaaaaaaaaaaaaggatccccgggcgagctcccaaaaaaaaaaaaaaaaaaaaaaaaaaaaaaccgaattcctgcagctcgagaaaaaaaaaaaaaaaaaaaaaaaaaaaaaaaaaaaaaaaaagatctggcgcgccgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacg

SEQ ID NO: 25 - protein, 758 aa, Homo sapiens
METDTLLLWVLLLWVPGSTGAASQVQLVQSGSELKKPGASVKVSCKASGYTFTNYGMNWVRQAPGQGLEWMGWINTYTGEPTYADDFKGRFVFSLDTSVSTAYLQISSLKAEDTAVYYCARWLRDFDYWGAGTTVTVSSGGGGSGGGGSGGGGSEIVLTQSPATLSLSPGERATLSCSASSSISYMHWYQQKPGQAPRLLIYDTSKLATGIPARFSGSGSGTDFTLTISSLEPEDFAVYYCHQRSSYPYTFGGGTKLEIKGGGGSGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSGGGGSGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSRENLYFQGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 217 aa, Homo sapiens
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0388695 A1
(43) Pub. Date: Dec. 25, 2025

(54) EPCAM-CD3 EPSILON BISPECIFIC ANTIBODIES

(71) Applicants: ProMab Biotechnologies, Inc., Richmond, CA (US); Forevertek Biotechnology Co., Ltd, Changsha (CN)

(72) Inventors: Lijun Wu, Berkeley, CA (US); Vita Golubovskaya, Pinole, CA (US)

(21) Appl. No.: 19/255,825

(22) Filed: Jun. 30, 2025

Related U.S. Application Data
(63) Continuation of application No. PCT/US2024/010201, filed on Jan. 3, 2024.
(60) Provisional application No. 63/478,380, filed on Jan. 4, 2023.
(60) Provisional application No. 63/508,205, filed on Jun. 14, 2023.

(51) Int. Cl. C07K 16/30; A61K 39/00; A61P 35/00; C07K 16/28

(52) U.S. Cl. CPC C07K 16/30; A61P 35/00; C07K 16/2809; A61K 2039/505; C07K 2317/14; C07K 2317/24; C07K 2317/31; C07K 2317/522; C07K 2317/524; C07K 2317/526; C07K 2317/53; C07K 2317/55

(57) ABSTRACT

The present invention is directed to bispecific humanized EPCAM CD3 epsilon chain (CD3e) antibodies. This invention provides different structures of EpCAM-CD3 antibody and provides methods of antibody production in cells through mRNA by in vitro transcription method. EpCAM-CD3 human Fc bispecific antibodies generated with mRNA-lipid nanoparticle (LNP) technology demonstrate high efficacy in vitro and in vivo.

### CROSS-REFERENCE TO RELATED APPLICATION(S)

This application is a continuation of PCT/US2024/010201, filed Jan. 3, 2024; which claims the benefit of U.S. Provisional Application Nos. 63/478,380, filed Jan. 4, 2023, and 63/508,205, filed Jun. 14, 2023. The contents of the above-identified applications are incorporated herein by reference in their entirety.

### REFERENCE TO SEQUENCE LISTING, TABLE OR COMPUTER PROGRAM

This application contains an ST.26 compliant Sequence Listing, which was submitted in xml format via Patent Center and is hereby incorporated by reference in its entirety. The .xml copy, created on Jan. 2, 2024, is named SequenceListing.xml and is 52400 bytes in size.

### FIELD OF THE INVENTION

The present invention relates to EPCAM-CD3 epsilon chain (CD3e) bispecific antibodies. The present invention is also directed to a method for killing EPCAM-positive cancer cells by administering EPCAM-CD3e bispecific antibody with T cells to the patients.

### BACKGROUND OF THE INVENTION

Immunotherapy is emerging as a highly promising approach for the treatment of cancer. T cells or T lymphocytes, the armed forces of our immune system, constantly look for foreign antigens and discriminate abnormal (cancer or infected cells) from normal cells. Using bispecific antibodies binding T cells and tumor associated antigen is the most common approach to design bispecific antibody by bringing cytotoxic T cells to kill cancer cells. Bispecific antibodies can be infused into patients by different routes. The advantage of bispecific antibodies compared with chemotherapy or antibody is that it specifically targets antigen-positive cancer cells and simultaneously activates T cells.

Redirecting the activity of T cells by bispecific antibodies against tumor cells, independently of their TCR specificity, is a potent approach to treat cancer. The concept is based on recognition of a cell surface tumor antigen and simultaneous binding to the CD3 epsilon chain (CD3e or CD3) within the T-cell receptor (TCR) complex on T cells. This triggers T-cell activation, including release of cytotoxic molecules, cytokines and chemokines, and induction of T-cell proliferation.

### EPCAM

EPCAM is an Epithelial Cell Adhesion Molecule that is encoded by EPCAM gene. EPCAM is a cell surface glycoprotein of approximately 40 kDa which is highly expressed in epithelial cancers and has lower expression in normal epithelial tissues (1), (2, 3). There are several names of EPCAM such as TROP1, CD326, HEA125, EGP40, KSA, ESA (1). EPCAM regulates cell-cell contact adhesions and tissue plasticity, and controls cell proliferation and differentiation (1),(4).

EPCAM shows high potential as a target for developing anticancer therapies and immunotherapies with monoclon NOal antibodies or bispecific antibodies.

Human EPCAM is a polypeptide of 314 amino acids, consisting of an extracellular domain (N-terminal) from 24-265 amino acids), a single-spanning transmembrane domain from 266 to 288 aa (underlined below) and a short cytoplasmic domain 289-314 amino acids (C-terminal) (1). The EPCAM sequence can be found in Uniprot database (www.uniprot.org/uniprot/P16422).

### BRIEF DESCRIPTION OF THE DRAWINGS

FIG. 1A shows EPCAM-CROSS-FAB-CD3 Knob in hole (KIH) CrossFAB format antibody. FIG. 1B shows EPCAM scfv-CD3 ScFv (BITE) format antibody. FIG. 1C shows EPCAM scFv-CD3 ScFv-Fc domain format antibody.

FIG. 2 shows the scheme of DNA vector template (top) used for in vitro transcription of RNA (bottom). 5′UTR, 5′ untranslated region; 3′UTR, 3′untranslated region; poly A tail for increased stability.

FIG. 3 shows EPCAM-CD3 with T cells antibody kills EPCAM-positive cells. RTCA assay was done using Lovo cells (structure of FIG. 1A).

FIG. 4 shows that PBM005 induced IFN-gamma secretion by T cells against EPCAM-positive Lovo cells in a dose-dependent manner. There was no induction of IFN-gamma secretion by T cells against EPCAM-negative HL-60 cells.

FIG. 5A shows EpCAM ScFv-CD3 ScFv antibody (PBM0065) with T cells secreted IFN-gamma in a dose-dependent manner with EPCAM-positive Lovo cells but not with HL-60 cells (structure of FIG. 1B). FIG. 5B shows the EC50 based on IFN-gamma secretion by EPCAM-CD3 antibody is 5.3 ng/ml (structure of FIG. 1B).

FIG. 6 shows EpCAM ScFv-CD3 ScFv-hinge-Fc antibody (PBM0070) with T cells secreted IFN-gamma in a dose-dependent manner with EPCAM-positive Lovo cells, but not with HL-60 cells (structure of FIG. 1C).

FIG. 7 shows the representative IFN-gamma ELISA assay with EC50=6.1 ng/ml (structure of FIG. 1C).

FIGS. 8A-8C show that EPCAM-CD3 antibody with T cells killed EPCAM-positive Lovo cells but not EPCAM-negative Colo 741 or Colo 320 cells. RTCA assay was performed either with T cells alone, antibody alone, or T cells plus different dilutions of antibody supernatant (structure of FIG. 1C).

FIG. 9 shows that EpCAM-CD3 antibody with t cells secreted IFN-gamma with EPCAM-positive Lovo cells, but not with EPCAM-negative Colo 741 and Colo 329 cells (structure of FIG. 1C).

FIG. 10 shows that EPCAM-CD3-hinge-Fc-LNP injected with T cells intratumorally into mice significantly decreased mouse xenograft tumor growth in vivo. OVCAR-5 cells were injected subcutaneously and RNA-LNP were (i) delivered intratumorally (i.t.) at days 1, 8, and 15 at dose 20 μL (4 ug) per tumor, or (ii) delivered intracellularly (i.c) on day 0, with OVACR-5 cells 20 μL (4 ug), and on day 16 with 2× dose 40 μL intratumorally (i.t.) per tumor. i.t shows intratumoral delivery; i.c+i.t. shows intracellular and intra-tumoral delivery.

FIGS. 11A-11B show that EpCAM-CD3 hFc mRNA-LNPs delivered to OVCAR-5 tumors with single intravenous injection of T cells significantly decreased OVCAR-5 xenograft tumor growth. A. The schedule for mRNA-LNPs intratumoral injection and a single injection of T cells by i.v delivered to OVCAR-5 xenograft tumor model. B. EpCAM-CD3 hFc mRNA-LNPs significantly decreased OVCAR-5 tumor growth combined with a single injection of T cells (structure of FIG. 1C).

### DETAILED DESCRIPTION OF THE INVENTION

### Definitions

As used herein, “affinity” is the strength of binding of a single molecule to its ligand. Affinity is typically measured and reported by the equilibrium dissociation constant (K_{D}), which is used to evaluate and rank order strengths of bimolecular interactions.

As used herein, “bispecific antibody” is an artificial protein that can simultaneously bind to two different types of antigen or different epitopes of the same antigen.

As used herein, “CD3 epsilon (CD3e)” is a polypeptide encoded by the CD3E gene which resides on chromosome 11 in human. CD3-epsilon polypeptide, which together with CD3-gamma, -delta and -zeta, and the T-cell receptor alpha/beta and gamma/delta heterodimers, forms the T cell receptor-CD3 complex. This complex plays an important role in coupling antigen recognition to several intracellular signal-transduction pathways. The CD3 epsilon polypeptide plays an essential role in T-cell development. CD3 epsilon, CD3e, and CD3 are used interchangeably in this application.

As used herein, a “domain” means one region in a polypeptide which is folded into a particular structure independently of other regions.

As used herein, a “single chain variable fragment (scFv)” means a single chain polypeptide derived from an antibody which retains the ability to bind to an antigen. An example of the scFv includes an antibody polypeptide which is formed by a recombinant DNA technique and in which Fv regions of immunoglobulin heavy chain (H chain) and light chain (L chain) fragments are linked via a spacer sequence. Various methods for preparing an ScFv are known to a person skilled in the art.

As used herein, a “tumor antigen” means a biological molecule having antigenicity, expression of which causes cancer.

The present invention is directed to bispecific antibodies that specifically binds to both human EPCAM and human CD3e. The EPCAM-CD3e bispecific antibody targets EPCAM tumor antigen which is highly overexpressed in many types of cancer such as ovarian, seminoma, and colon cancer. The EPCAM-CD3 bispecific antibodies of the present invention have high cytotoxic activity EPCAM-positive colon cancer cell line and don't have activity with EPCAM-negative cell line. The bispecific antibody activates T cells and re-directs T cells to EPCAM-positive cancer cells.

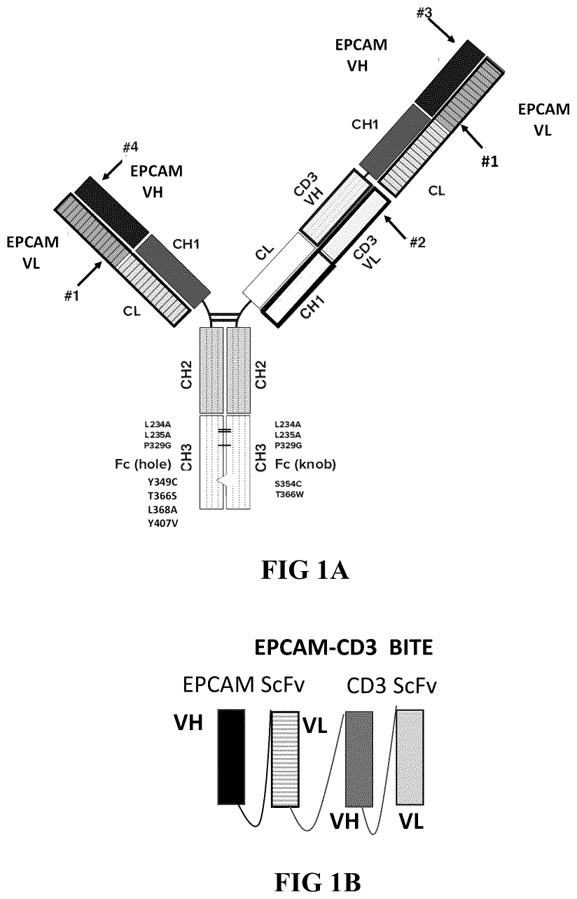

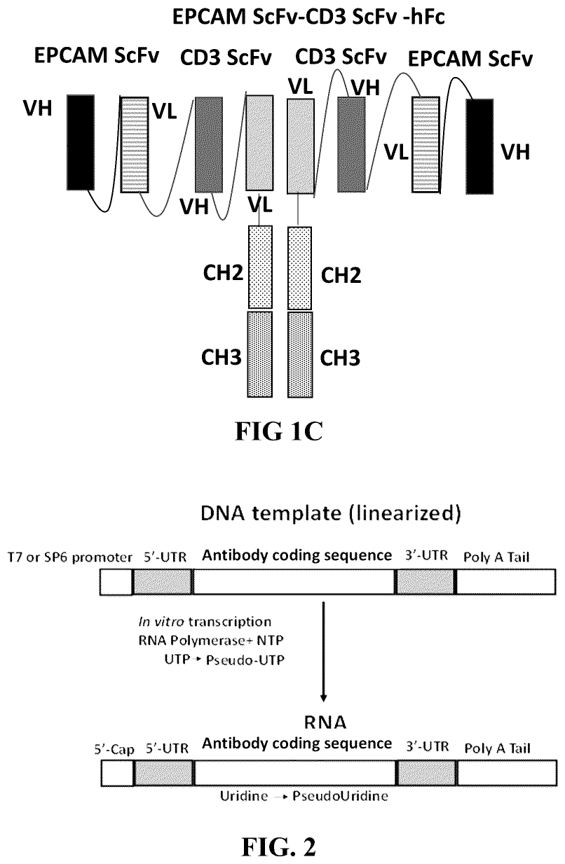

Three bispecific antibody structures of the present invention are shown in FIGS. 1A-1C. FIG. 1A shows a heterodimeric knob-in hole CrossFab antibody that binds with one arm to human CD3e chain expressed on T cells and with two arms to human EPCAM expressed on EPCAM-positive cancer cells. FIG. 1B shows a BITE antibody that binds with one arm to human CD3e chain and one arm to human EPCAM antigen. FIG. 1C shows antibody that has EPCAM ScFv-CD3 ScFv with human Fc for stability.

FIGS. 1A-1C show the structures of bi-specific humanized EPCAM and CD3 antibodies.

FIG. 1A shows bivalent EpCAM-CrossFAB-CD3 knob in hole CrossFab format. The knobs-in-hole structure and silent Fc mutations P329G and leucine to alanine (L234A, L235A or LA-LA) mutations are shown in structures FIG. 1A. The amino acid numbers in CH3 are counted from human IgG1 according to [6]. FIG. 1B shows DNA construct encoding one polypeptide of humanized EPCAM-CD3 BITE antibody. FIG. 1C shows DNA construct encoding one polypeptide of humanized EPCAM-CD3-hFc antibody. The antibody of FIG. 1A have two EPCAM binding moieties and one CD3 binding moiety. The antibody of FIGS. 1B and 1C has one EPCAM binding moiety and one CD3 binding moiety and either have BITE format (B) or have dimeric human Fc (C).

### Bispecific Antibody Structure of FIG. 1A

In one aspect, the present invention is directed to a bispecific antigen-binding molecule having structure of FIG. 1A (EpCAM-CD3 CrossMab knob-in-hole). In one aspect, the EPCAM antibody is a humanized antibody, and the bispecific antibody comprises: (a) a first and a second antigen-binding moiety each of which is a humanized Fab molecule capable of specific binding to human EPCAM, and each comprises a heavy chain variable region (EPCAM VH), for example, having the amino acid sequence of SEQ ID NO: 10, and a light chain variable region (EPCAM VL), for example, having the amino acid sequence of SEQ ID NO: 4; (b) a third antigen-binding moiety which is a Fab molecule capable of specific binding to human CD3 epsilon, the third antigen-binding moiety comprises a heavy chain variable region (CD3 VH), for example, having the amino acid sequence of SEQ ID NO: 11, and a light chain variable region (CD3 VL), for example, having the amino acid sequence of SEQ ID NO: 7, wherein the third antigen-binding moiety is a crossover Fab molecule, in which the constant regions of the Fab light chain and the Fab heavy chain are exchanged; and (c) an human IgG Fc domain comprising a first subunit and a second subunit capable of stable association; wherein the Fab heavy chain of the third antigen-binding moiety is (i) fused at the N-terminus to the C-terminus of the Fab heavy chain of the first antigen-binding moiety (CH1), and (ii) fused at the C-terminus to the N-terminus of the first subunit of the Fc knob domain, and wherein the second antigen-binding moiety is fused at the C-terminus of the Fab heavy chain (CH1) to the N-terminus of the second subunit of the Fc hole domain.

The bispecific antibody of the present invention uses CROSSFAB approach, which crossovers the constant domain and variable domain and switches the CH1 domain and CL domain in the CD3e Fab molecule, which reduces undesired mis-paring.

In one embodiment, the bispecific antibody of the present invention comprises: (1) humanized EPCAM light chain, (2) CD3e cross FAB, CD3 VL-CH1; (3) humanized EPCAM VH-CH1-CD3e CROSSFAB (VH-CL)-Fc (knob), and (4) humanized EPCAM VH-CH1-Fc (hole). (FIG. 1A)

As shown in FIG. 1A, human IgG Fc contains a hinge, CH2 and CH3.

In one embodiment, the VH of the humanized EPCAM antibody has the amino acid sequence of SEQ ID NO: 10 and the VL has the amino acid sequence of SEQ ID NO: 4.

In one embodiment, the Fc domain comprises a modification promoting the association of the first and the second subunit of the Fc domain.

In one embodiment, in the CH3 domain of the first subunit of the Fc domain, an amino acid residue is replaced with an amino acid residue having a larger side chain volume, thereby generating a protuberance within the CH3 domain of the first subunit which fits in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit fits.

In one embodiment, the Fc domain exhibits reduced binding affinity to an Fc receptor and/or reduced effector function, as compared to a native IgG Fc domain.

In one embodiment, the Fc domain comprises one or more amino acid substitution that reduces binding to an Fc receptor and/or effector function. In one embodiment, the one or more amino acid substitutions in the Fc domain are selected from the group of L234, L235, and P329 (Kabat numbering). In one embodiment, said amino acid substitutions are L234A, L235A and P329G.

In one embodiment, Fc mutations P329G, and L234A and L235A mutations are used to prevent Fc-dependent immune reactions.

In one embodiment, only mutations L234A and L235A mutations are used to prevent Fc-dependent immune reactions.

In a specific embodiment, the Fc domain is modified with a so-called “knob-into-hole” modification, comprising a “knob” modification in one of the two subunits of the Fc domain and a “hole” modification in the other one of the two subunits of the Fc domain. The knob-into-hole technology is described e.g. in U.S. Pat. No. 5,731,168. Generally, the method involves introducing a protuberance (“knob”) at the interface of a first polypeptide and a corresponding cavity (“hole”) in the interface of a second polypeptide, such that the protuberance can be positioned in the cavity to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

In one embodiment, a “knob” is made by mutations of S354C and T366W on one Fc, and the corresponding “hole” is made by mutations of Y349C, T366S, L368A and Y407V on the partner Fc.

In one embodiment, the bispecific antigen-binding molecule comprising two binding moieties to EPCAM, and one binding moiety to CD3 epsilon, the molecules comprises the amino acid sequences of SEQ ID NO: 5, 8, 12, and 14, in a molar ratio of 2:1:1:1.

### Bispecific Antibody Structure of FIG. 1B

In one aspect, the present invention is directed to a bispecific antigen-binding molecule having structure of FIG. 1B (EpCAM ScFv-CD3 ScFv (BITE)).

FIG. 1B shows the structure of humanized bispecific EPCAM ScFv-CD3e ScFv antibody consisting of one DNA constructs. This BITE structure comprises one binding moiety to EPCAM and one binding moiety to CD3 epsilon and has 6xHis tag at the end for purification.

The present invention is directed to a bispecific antigen-binding molecule comprising EPCAM VH, for example, having SEQ ID NO: 10, a first linker, EPCAM VL, for example, having SEQ ID NO: 4, a second linker, CD3 VH, for example, having SEQ ID NO: 11, a third linker, CD3 VL, for example, having SEQ ID NO: 7, wherein the first, the second, and the third linkers are the same or different.

### Bispecific Antibody Structure of FIG. 1C

In one aspect, the present invention is directed to a bispecific antigen-binding molecule having structure of FIG. 1C (EpCAM ScFv-CD3 ScFv-human Fc domain).

FIG. 1C shows a bispecific antibody structure of monovalent humanized EPCAM ScFv and monovalent CD3e ScFv fused to a human Fc domain; the structure consists of one DNA construct. The structure has an EPCAM scFv and CD3e scFv connected by a linker, and then a Fc domain (a hinge linker-CH2-CH3) fused to CD3 Scfv to increase stability. The bispecific antibody (BITE-human Fc format) comprises one binding moiety to EPCAM, and one binding moiety to CD3 epsilon. The antibody may have a dimeric conformation.

The present invention provides a bispecific antigen-binding molecule comprising humanized EPCAM VH, a first linker, humanized EPCAM VL, a second linker, CD3 VH, a third linker, CD3 VL, and a Fc domain.

In one embodiment, the EPCAM VH has the amino acid sequence of SEQ ID NO: 10, the EPCAM VL has the amino acid sequence of SEQ ID NO: 4, the CD3 VH has the amino acid sequence of SEQ ID NO: 11, and the CD3 VL has the amino acid sequence of SEQ ID NO: 7.

In one embodiment, the Fc domain comprises a hinge linker, and CH2-CH3 of human IgG1, optionally substituted with one or two amino acid substitutions.

In one embodiment, the Fc domain comprises one or more amino acid substitutions selected from the group of L234A, L235A, and P329G (EU numbering).

In one embodiment, each first, second, and third link has the amino acid sequence of (GGGGS)n, and n=1-5. In one preferred embodiment, the linker has the amino acid sequence of (GGGGS)₃.

In one embodiment, the bispecific antigen-binding molecule has the amino acid sequence of SEQ ID NO: 19.

### Method for Preparing Bispecific Antibody

The nucleic acid encoding an antibody or an antigen-binding fragment thereof can be inserted into a vector and expressed in mammalian 293S or CHO cells using serum-free medium. The antibody with human Fc can be purified with protein A or protein G column and used for the study. The antibody with His tag can be purified with single-step affinity chromatography, namely immobilized metal ion affinity chromatography (IMAC), which is commercially available in different kinds of formats, Ni-NTA matrices being the most widely used.

The present invention provides an isolated DNA sequence comprising (a) a promoter coding sequence, (b) 5′-UTR (untranslated region) coding sequence, (c) a coding sequence to encode an antibody or an antigen-binding fragment thereof, (d) a 3′-UTR coding sequence, and (e) a poly A tail sequence. In one embodiment, the antibody or the antigen-binding fragment thereof is the bispecific antigen-binding molecule of the present invention.

In the DNA sequence, the promoter is T7, T7AG promoter, or SP6 promoter. Poly A tail sequence is from 20-170 nucleotides. Poly A tail sequence optionally comprises one or more linkers in between the poly A segments. If poly A tail is longer than 60 nucleotides, than it typically contains a linker which includes non-adenosine nucleotides. A linker is 5-30 or 5-25 nucleotides, e.g., 10 nucleotides or 20 nucleotides. In one example, poly A tails is 110 nucleotides in length, consisting of a stretch of 30 adenosine residues, followed by a 10-nucleotide linker sequence, and another 70 adenosine residues. In another example, poly A tails is 90 nucleotides in length, consisting of a stretch of 40 adenosine residues, followed by a 30-nucleotide linker sequence, and another 30 adenosine residues. In yet another example, poly A tail is 150-160 nucleotides in length, consisting of a two linker sequences.

DNA expression is finely regulated at the post-transcriptional level. Untranslated regions are not translated into amino acids, however, UTRs of mRNAs may control their translation, degradation and localization include stem-loop structures, upstream initiation codons and open reading frames, internal ribosome entry sites and various cis-acting elements that are bound by RNA-binding proteins. UTRs are important in the post-transcriptional regulation of DNA expression, including modulation of the transport of mRNAs out of the nucleus and of translation efficiency, subcellular localization, and stability.

5′-UTR typically has 10-1000 nucleotides, or 20-500 nucleotides, or 30-200 nucleotides, or 30-100 nucleotides. For example, 5′-UTR is 50 nucleotides. 3′-UTR typically has 10-3000 nucleotides, for example, 50-500 nucleotides, or 100-300 nucleotides. Preferred 5′-UTRs and 3′-UTRs are UTRs of β-globin, or UTRs of Pfizer COVID vaccine.

β-Globin gene is shown in:

https://www.ncbi.nlm.nih.gov/nucleotide/V00497.1?report=genbank&log$=nuclalign&blast_ran k=5&RID=TDDZ1K98016

In one embodiment, the 5′-untranslated region is derived from human alpha-globin RNA with an optimized Kozak sequence. The 3′ untranslated region comprises two sequence elements derived from the amino-terminal enhancer of split (AES) mRNA and the mitochondrial encoded 12S ribosomal RNA to confer RNA stability and high total protein expression.

Any suitable vector, such as Vector pSP64 Poly(A) (Promega) or pGEM3Z-Vector (Promega) can be used as a cloning vector for the DNA sequence described above.

For example, to engineer the pEM3Z-β-globin UTR-UTR-poly A tail, the 3′-UTR of the β-globin molecule flanked by restriction enzyme site can be amplified from human bone marrow. For example, a single (pEM3Z-1β-globin-UTR-A[120]) or 2 serial fragments (pEM3Z-2β-globin-UTR-A[120]) can be inserted in front of the poly(A) tail.

The present invention provides a method for producing an antibody or an antigen-binding fragment thereof in cells and, also in vivo inside an animal. The method comprises the steps of: obtaining the DNA sequence as described above, transcribing the DNA sequence to mRNA with RNA polymerase in vitro, electroporating or transfecting the mRNA into cells or into tumors, and translating the mRNA in the cells to produce the antibody or an antigen-binding fragment thereof. In one embodiment, the antibody or the antigen-binding fragment thereof is the bispecific antigen-binding molecule of the present invention.

FIG. 2 shows the scheme of DNA vector template (top) used for in vitro transcription of RNA (bottom). 5′UTR, 5′ untranslated region; 3′UTR, 3′untranslated region; poly A tail for increased stability.

The present invention is also directed to a pharmaceutical composition comprising the bispecific antigen-binding molecule and a pharmaceutically acceptable carrier.

The inventors demonstrated that EpCAM-CD3 antibody can be produced using in vitro transcription of DNA template to produce antibody using RNA transfected into 293 cells.

Antibodies also can be produced using adenoviruses or other viruses inside the cells providing in vivo manufacturing which decreases cost of manufacturing, generating stable cell lines for production of antibodies.

In one embodiment, antibody DNAs are inserted into DNA template vector with either T7 or SP6 promoter for RNA polymerase to transcribe to antibody RNAs by in vitro transcription. RNA is mixed with lipid components to produce lipid nanoparticles (LNPs) with RNA encapsulated. Then LNP-encapsulated mRNAs are electroporated or transfected into either T cells, primary NK cells, NK-92, DC, macrophages or other types of immune cells or mammalian cells to translate mRNAs inside cells to produce antibody protein, which can be used for killing of tumor cells.

In general, mRNA is transient and short-lived when delivered in vivo. The present invention provides a method for producing an antibody or antigen-binding molecule by delivering lipid nanoparticle-encapsulated mRNA of an antibody or an antigen-binding molecule in cells. By encapsulating mRNA in lipid nanoparticles, the stability of mRNA is improved. The method comprises the steps of: (i) obtaining a DNA sequence comprising: (a) a promoter coding sequence, (b) 5′-UTR (untranslated region) coding sequence, (c) a sequence to encode an antigen-binding molecule, (d) a 3′-UTR coding sequence, and (e) a poly A tail sequence; (ii) transcribing the DNA sequence to mRNA with RNA polymerase in vitro, (iii) encapsulating the mRNA in lipid nanoparticles (LNPs), (iv) transfecting the mRNA-encapsulated LNPs into cells, and (v) translating the mRNA in the cells to produce the antigen-binding molecule.

In one embodiment, the lipid nanoparticles comprise 8-[(2-hydroxyethyl)[6-oxo-6-(undecyloxy)hexyl]amino]-octanoic acid, 1-octylnonyl ester (SM-102), distearoylphosphatidylcholine (DSPC), Cholesterol, and 1,2-dimyristoyl-rac-glycero-3-methoxypolyethylene glycol-2000 (DMG-PEG2000). [LNP-102 (ii)]

In one embodiment, the lipid nanoparticles comprise 8-[(2-hydroxyethyl)[6-oxo-6-(undecyloxy)hexyl]amino]-octanoic acid, 1-octylnonyl ester (SM-102), distearoylphosphatidylcholine (DSPC), Cholesterol, and 1,2-distearoyl-sn-glycero-3-phosphoethanolamine-N-[maleimide(polyethylene glycol)-2000] (DSPE-PEG2000-MAL). [LNP-102 (i)]

In one embodiment, the lipid nanoparticles comprise 2-hexyl-decanoic acid, 1,1′-[[(4-hydroxybutyl)imino]di-6,1-hexanediyl] ester (ALC-0315), DSPC, Cholesterol, and α-[2-(ditetradecylamino)-2-oxoethyl]-ω-methoxy-poly(oxy-1,2-ethanediyl) (ALC-0159). [LNP-315]

Insertion of mRNA into LNP nanoparticles provides protection of mRNA from degradation and increases the stability mRNA; mRNA is then released from LNPs into cells in vivo to generate protein. mRNA-lipid nanoparticle preparation is described in Schoenmaker (International J. Pharmaceutics, 601: 120856, 2021), the article is incorporated herein by reference in its entirety, in particular regarding the LNPs.

### Method for Treating Cancer

The present invention is directed to a method for treating cancer, comprising the step of administering a bispecific EPCAM-CD3e antigen-binding molecule or antibody to a subject suffering from cancer by intratumor injection, wherein the cancer is selected from the group consisting of colon cancer, lung cancer, pancreatic cancer, stomach cancer, testicular cancer, teratoma, seminoma, ovarian cancer, and cervical cancer, and the cancer is EPCAM-positive.

In one embodiment, the method comprises the steps of: obtaining an mRNA sequence comprising a coding sequence to encode a bispecific EPCAM-CD3e antigen-binding molecule; mixing the mRNA with lipid nanoparticles to form a mRNA-lipid nanoparticle complex; and injecting the mRNA-lipid nanoparticle complex into cancer cells.

In one embodiment, the bispecific EPCAM-CD3e antigen-binding molecule has a sequence as described above in this application.

In one embodiment, the method further comprises injecting T cells intravenously.

This application demonstrates the efficacy of bispecific antibody targeting EPCAM antigen that is overexpressed in colon cancer tumors. This application demonstrates that EPCAM-CD3e antibody binds CD3e antigen and EPCAM antigen. This antibody delivered with T cells specifically decreases viability of EPCAM-positive colon cancer cells but not EPCAM-negative cancer cells. EPCAM-CD3e antibody delivered with T cells caused secretion of significant level of IFN-gamma after co-incubation with EPCAM-positive colon cancer cells but not after co-incubation with EPCAM-negative target cancer cells. This application demonstrates that EPCAM-CD3e antibody administered with T cells significantly decreased Lovo (positive EPCAM-colon cancer cells) tumor growth in vitro.

In one embodiment, RNA is embedded into LNPs and transfected into normal cells, cancer cells, and inside tumors in vivo. The nanoparticle-based drug delivery demonstrated many advantages, such as high bioavailability, solubility, stability, passage through the blood-brain barrier, and low toxicity and minimal side effects.

The inventors demonstrate that EPCAM-CD3 antibody with T cells significantly killed all EPCAM-positive cancer cells, but not kill EPCAM-negative colon cancers. This implies high specificity of EPCAM-CD3 antibody.

The inventors demonstrate high efficacy of three different designs of bispecific antibodies of FIGS. 1A-1C.

The inventors demonstrated that EPCAM-CD3-hFc RNA-LNP transfected to cancer cell lines can produce antibody.

In addition, RNA template with EPCAM bispecific antibody can have human Fc can include silent mutations such as L234A, L235A, P329G to decrease Fc-dependent immune response, ADCC.

The RNA template with EpCAM bispecific antibody also can be made without Fc for shorter sequence and shorter half-life on antibody.

The inventors demonstrate that EPCAM-CD3-hFc antibody RNA-LNP injected into OVACR-5 tumors with T cells significantly decreased xenograft tumor growth.

This application shows that in vivo production of antibody from injected RNA-LNP can provide high in vivo efficacy.

In the examples below, three different designs of humanized EpCAM-CD3 antibodies were engineered and tested against colorectal tumors. The antibodies demonstrated high efficacy and specificity. In addition, the study demonstrates a method of delivering bispecific antibodies using mRNA-lipid nanoparticle (LNP) technology. The delivery of EpCAM-CD3 human Fc (hFc) mRNA-LNPs into mice tumors with intravenous T cell injection significantly blocked OVCAR-5 xenograft tumor growth in vivo.

The production of bispecific antibodies through mRNA-LNP injection in vivo has many advantages versus RNA or antibody proteins. The mRNA-LNP complexes are more stable than regular mRNA, and the production of antibodies through mRNA-LNPs is less costly than antibody protein manufacturing and shows potential for further optimizations in future studies. Intratumoral delivery of EpCAM-CD3 hFc mRNA-LNPs with intravenous delivery of T cells blocked xenograft tumor growth. This approach can be used against solid tumors.

The present disclosure describes a safe local intratumoral delivery using mRNA-LNP technology to deliver bispecific antibody. The present disclosure combines a cell therapy approach by using T cells to kill tumors. This approach can be used similarly to attract NK cells or gamma-delta T cells to tumors using specific immune cell receptors. In addition, different stimulators of immune cells can be used to increase the efficacy of this therapy such as checkpoint inhibitor players, chemokines, cytokines, growth factors, and tumor microenvironment modulators. The lysed tumor releases neoantigens (natural vaccine), which can be recognized in the presence of immunomodulators by antigen-presenting cells or dendritic cells and promote the activity of memory T and B cells.

The following examples further illustrate the present invention. These examples are intended merely to be illustrative of the present invention and are not to be construed as being limiting.

### EXAMPLES

### Example 1. Materials and Methods

### Cells and Culture Medium

HEK293FT cells from AlStem (Richmond, CA) were cultured in Dulbecco's Modified Eagle's Medium (DMEM) plus 10% FBS and 1% penicillin/streptomycin. Human peripheral blood mononuclear cells (PBMC) were isolated from whole blood obtained from the Stanford Hospital Blood Center, Stanford, CA according to IRB-approved protocol using Ficoll-Paque solution (GE Healthcare). Colon cancer cell lines: EPCAM-negative: Colo-741 and Colo-320 and EPCAM-positive: Lovo cells were used for the study. The cells were cultured in a humidified 5% CO₂ (5).

### Antibodies

Allophycocyanin-(APC) labelled antihuman CD326 (EpCAM) antibody was obtained from Biolegend. PE-conjugated anti-His tag antibody was from Biolegend. APC-labelled antihuman CD3 antibody was obtained from Biolegend. Antihuman IgG was from Jackson ImmunoResearch. PE and 7-AAD viability staining solution was obtained from Biolegend

### EPCAM-CD3 Antibody Constructs

The four constructs of Example 2 were designed according to Cross-Fab designed described in (6). The constructs had P329G mutation and Leucine 234,235 changed to alanine, called LA-LA to decrease Fc immune activity. In addition, Fc silent and knobs-in-hole mutations were used for engineering, as described (6). We also expressed two constructs shown on FIG. 1B and FIG. 1C. All constructs for FIG. 1A were cloned into Nhe I and Nsi I sites of pYD11 vector.

### Design and Cloning of Bispecific Antibody DNA Constructs

The AUA1 antibody was produced conventionally by immunizing BALB/c mice with the colon adenocarcinoma cell line Lovo. The mouse AUA1 EPCAM antibody was characterized. Humanization of the mouse AUA1 antibody (7) was performed as described (8, 9). The humanized antibody was first checked for functional activity in the chimeric antigen receptor (CAR) format as described (9). The humanized variable fragment heavy chain (VH) and light fragment chain (VL) of the AUA1 antibody were used for the engineering of CrossMab knob-in-hole design bivalent antibody constructs as described (10). Human Fc (IgG1) contained P329G and L234AL235A (LALA) mutations to silence the Fc region by preventing the binding of gamma receptors and activating innate immune cells.

Another design contained an EpCAM ScFv-CD3 ScFv-His tag (BITE) format.

The third design was an EpCAM ScFv-CD3 ScFv-human Fc construct.

All constructs were cloned into the pYD11 vector and confirmed by sequencing. For the design of DNA templates for RNA-based expression, the EpCAM ScFv-CD3 ScFv-human Fc sequence was cloned into a DNA vector with a T7AG promoter, 50UTR, 30UTR, and a 150 poly A tail as described (11). The DNA template sequence for in vitro transcription was verified by sequencing.

### Expression of EPCAM-CD3 Antibodies

For structure FIG. 1A, the four antibody constructs were mixed at weight ratio 2 (EPCAM VL-CL):1:1:1 (μg/mL) with NanoFect transfection agent and used for 293S cell transformation. For structures 1B and 1C, the antibody constructs were mixed at weight ratio with NanoFect transfection agent and used for 293S cell transformation. The cells were rotated in bottles on shaker in Freestyle F17 medium, containing 8 mM L-Glutamine (or GlutaMAX), and 0.1% Pluoronic F-68 for one week at 37° C. incubator. The supernatant or purified antibody with protein A column for Fc-containing antibodies (FIG. 1A, FIG. 1C) or with Ni-NTA column for His-tagged (FIG. 1B) antibody was analyzed on SDS gel, by FACS and functional assays.

### Transfection of HEK-293 Cells With DNA Encoding Bispecific Antibodies

HEK293S cells were transfected using DNA constructs of the bispecific antibodies with the ALSTEM NanoFect Transfection Reagent. The cells were cultured in Freestyle F17 medium with 8 mM glutamine and 0.1% Pluronic F68 surfactant in suspension bottles using a shaker at 37° C. and 5% CO₂. Supernatants containing antibodies were collected on days 3-7 post-transfection. The supernatants containing antibodies with human Fc were purified using protein A or protein G columns. The supernatant containing EpCAM-CD3-His tag antibody was purified using Ni-NTA columns. The purified antibodies were checked for size on SDS gel and then used for functional analyses.

### PBMC

PBMC were resuspended at 1×10⁶ cells/ml in AIM V-AlbuMAX medium (Thermo Fisher) containing 10% FBS with 300 U/ml IL-2 (Thermo Fisher). PBMC cells were activated with CD3/CD28 Dynabeads (Invitrogen) and used for cytotoxicity analysis with bi-specific antibodies.

### Fluorescence-Activated Cell Sorting (FACS) Analysis

The allophycocyanin (APC)-labeled anti-CD3 (eBioscience, San Diego, CA) antibody was used for FACS analysis using FACSCalibur (BD Biosciences). For FACS with colon cancer cell lines to detect EPCAM levels, either bi-specific EPCAM-CD3 or mouse monoclonal AUA1 antibody from Oxford University or from Biolegend (Biolegend Ant-hu-EpCAM-APC (Cat. No: 324207) were used and analyzed on FACSCalibur, as described (5).

### Real-Time Cytotoxicity Assay (RTCA)

Adherent colon cancer target cells (10,000 cells per well) were seeded into 96-well E-plates (Acea Biosciences, San Diego, CA) and cultured overnight using the impedance-based real-time cell analysis (RTCA) iCELLigence system (Acea Biosciences). After 20-24 hours, the medium was replaced with 1×10⁵ effector cells T cells, T cells with bispecific antibody or antibody alone in AIM V-AlbuMAX medium containing 10% FBS, in triplicate. The cells were monitored for >40 hours with the RTCA system, and impedance (proportional to cell index) was plotted over time. Cytotoxicity was calculated as (impedance of target cells without effector cells−impedance of target cells with effector cells)×100/impedance of target cells without effector cells.

### ELISA Assay for IFN-Gamma Cytokine Secretion

The target cells were cultured with the effector cells or agents at in U-bottom 96-well plates with AIM V-AlbuMAX medium plus 10% FBS, in triplicate. After 16 h the supernatant was removed and centrifuged to remove residual cells. In some experiments, supernatant after RTCA assay was used for IFN-gamma ELISA cytokine assay. The supernatant was transferred to a new 96-well plate and analyzed by ELISA for human cytokines using kits from Thermo Fisher according to the manufacturer's protocol. The EC50 was calculated with GraphPad Prism software.

### Example 2. The Sequence Of Humanized EPCAM-CD3E Bispecific Antibody (FIG. 1A)

FIG. 1A shows the structure of humanized EPCAM-CD3 bivalent antibody consisting of 4 DNA constructs. The structure has CD3 CROSS-Fab, called PBM005.

EPCAM-CD3e bispecific antibody of FIG. 1A comprises 4 constructs:
1. Humanized EPCAM light chain (VL-CL): EPCAM VL (PCT/US20/14999), and codon optimized as below)
2. CD3 CROSSFAB, (VL-CH1)
3. Humanized EPCAM VH-CH1-CD3 CROSSFAB (VH-CL)-Fc (knob) P329GLA-LA (EPCAM VH from PCT/US20/14999 and codon optimized as below)
4. Humanized EPCAM VH-CH1-Fc(hole) P329GLA-LA (EPCAM VH PCT/US20/14999 and codon optimized as below)

P329G mutation abolishes interaction of FcγR and C1q interactions and thus eliminates elimination of targeted cells via antibody-dependent cellular-cytotoxicity (ADCC), antibody-dependent phagocytosis (ADCP) or complement-dependent cytotoxicity (CDC). P329G mutation removes FcγR-mediated immune effector functions when delivered to cells providing silent Fc region (7). Addition of two other mutations LA-LA mutation changes Leu 234 and Leu 235 to alanine (A), which completely blocks binding of FcγR and C1q interactions and thus abolishes Fc-mediated ADC, ADCC and other immunogenicity (6).

All sequences were codon optimized and synthesized as GBlocks and inserted into Nhe I and Nsi I site of pYD11 vector. In order not to have mispairing of light chain domains, CrossFAB technology was used where CD3 VH is connected to CL, and CD3 VL is connected to CH1. We also use knobs-in-hole mutations proposed by Crick in 1952 to create the knob T366W and S354C, and the hole (Y349C, T366S, L368A and Y407V), which hold both Fc chains together. All sequences start with the signaling peptide (underlined):

Nucleotide sequence of signaling peptide:

(SEQ ID NO: 1)
ATGGAGACAGACACACTCCTGCTATGGGTACTGCTGCTCTGGGTTCCA
GGTTCCACTGGC

Amino acid sequence of signaling peptide:

(SEQ ID NO: 2)
METDTLLLWVLLLWVPGSTGAAS

### Construct #1. EPCAM Light Chain: LC-EPCAM

DNA artificial sequence LC (light chain) of humanized EPCAM (EPCAM VL (bold)-CL (italics) is shown below. The nucleotide sequence of EPCAM VL shown in WO2020/154627 was codon-optimized and inserted with constant CL region into Nhe I (GCTAGC site shown in italics, underlined) and Nsi I sites (atgcat shown in italics, underlined of pYD11 vector). The sequences started with signaling peptide (Signaling peptide is underlined, plus AAS amino acids due to cloning site):

(SEQ ID NO: 2)
METDTLLLWVLLLWVPGSTGAAS.

The stop codon was added to the sequence before start of human Fc to express light chain without Fc present in the vector. Signaling peptide in bold italics, underlined; VL bold; CL italics.

(SEQ ID NO: 3)
ATGGAGACAGACACACTCCTGCTATGGGTACTGCTGCTCTGGGTTCCAGGTTCCACTGGCGCCG
CTAGC
GAAATTGTGCTGACCCAGAGCCCGGCGACCCTGAGCCTGAGCCCGGGCGAACG
CGCGACCCTGAGCTGCAGCGCGAGCAGCAGCATTAGCTATATGCATTGGTATC
AGCAGAAACCGGGCCAGGCGCCGCGCCTGCTGATTTATGATACCAGCAAACTG
GCGACCGGCATTCCGGCGCGCTTTAGCGGCAGCGGCAGCGGCACCGATTTTAC
CCTGACCATTAGCAGCCTGGAACCGGAAGATTTTGCGGTGTATTATTGCCATCA
GCGCAGCAGCTATCCGTATACCTTTGGCGGCGGCACCAAACTGGAAATTAAACG
T ACA GTA GCT GCT CCG TCC GTC TTT ATT TTC CCG CCG TCT GAC GAA CA
G CTC AAA AGC GGG ACT GCATCA GTT GTC TGT CTC CTC AAC AAT TTT TAC
CCG CGA GAG GCT AAG GTT CAA TGG AAA GTT GAT AAC GCCCTC CAG AGT
GGA AAC TCT CAG GAG AGT GTA ACT GAG CAA GAT TCC AAA GAT TCA ACC
TAT AGT CTT TCAAGT ACC TTG ACT CTT TCT AAA GCG GAT TAT GAG AAA C
AT AAA GTG TAT GCC TGC GAA GTG ACC CAT CAGGGG CTT TCA TCA CCC GT
G ACG AAG TCC TTT AAC CGA GGC GAA TGC TAA.

### Amino Acid Sequence

Signaling peptide (underlined) + AAS:
(SEQ ID NO: 2)
METDTLLLWVLLLWVPGSTGAAS
HUMANIZED EPCAM VL
(SEQ ID NO: 4)
EIVLTQSPATLSLSPGERATLSCSASSSISYMHWYQQKPGQAPRLLIYD
TSKLATGIPARFSGSGSGTDFTLTISSLEPEDFAVYYCHQRSSYPYTFG
GGTKLEIK
EPCAM VL-CL
(SEQ ID NO: 5)
EIVLTQSPATLSLSPGERATLSCSASSSISYMHWYQQKPGQAPRLLIYD
TSKLATGIPARFSGSGSGTDFTLTISSLEPEDFAVYYCHQRSSYPYTFG
GGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC

### Construct #2. CD3 CROSSFAB (VL-CH1)

CD3 VL is shown in bold, CH1 is in italics font, the nucleotide sequence was codon optimized. The Nhe I and Nsi I sites are shown in italics. The stop codon TAA was added to terminate the sequence before Fc.

Nucleotide sequence: Signaling peptide underlined in italics in bold, then AAS in italics regular font; VL in bold, CH1, regular font italics.

(SEQ ID NO: 6)
ATGGAGACAGACACACTCCTGCTATGGGTACTGCTGCTCTGGGTTCCAGGTTCCA
CTGGCGCCGCTAGCCAG GCC GTA GTG ACA CAG GAA CCG TCT TTG A
CG GTG TCT CCG GGA GGT ACC GTC ACC TTG ACG TGT GGGTCC A
GC ACT GGA GCT GTA ACA ACG AGC AAT TAC GCG AAT TGG GTG
CAG GAG AAG CCA GGT CAG GCT TTTAGG GGT CTT ATC GGA GGG
ACT AAT AAA AGG GCT CCA GGC ACG CCG GCA AGA TTC TCA GGG
TCC CTG CTGGGG GGG AAA GCG GCA CTC ACC CTT TCT GGT GCT
CAG CCA GAG GAT GAG GCC GAA TAT TAT TGT GCC TTGTGG TAT
TCT AAT TTG TGG GTC TTT GGA GGC GGG ACA AAA CTC ACT GT
A TTG TCA TCT GCG TCA ACG AAGGGA CCT TCT GTA TTC CCC TT
G GCA CCA TCC AGT AAA TCT ACC AGT GGG GGT ACC GCT GCC C
TC GGT TGCCTT GTA AAA GAT TAC TTT CCG GAG CCC GTC ACC G
TG TCC TGG AAC AGC GGG GCA TTG ACC AGT GGT GTCCAC ACT T
TT CCC GCA GTA CTC CAA AGC TCC GGC CTC TAC AGT CTC TCT
TCA GTT GIG ACG GTT CCT AGCICT TCC CTT GGT ACG CAG ACT
TAT ATC TGC AAC GTC AAC CAC AAA CCT TCC AAT ACT AAG GTA
GAC AAAAAG GTG GAG CCC AAA TCT TGT TAAATGCAT

### Amino Acid Sequence (Not Including Signaling Peptide)

CD3 VL
(SEQ ID NO: 7)
QAVVTQEPSLTVSPGGTVTLTCGSSTGAVTTSNYANWVQEK
PGQAFRGLIGGTNKRAPGTPARFSGSLLGGKAALTLSGAQP
EDEAEYYCALWYSNLWVFGGGTKLTVL
CD3 VL-CH1
(SEQ ID NO: 8)
QAVVTQEPSLTVSPGGTVTLTCGSSTGAVTTSNYANWVQEK
PGQAFRGLIGGTNKRAPGTPARFSGSLLGGKAALTLSGAQP
EDEAEYYCALWYSNLWVFGGGTKLTVLSSASTKGPSVFPLAP
SSKSTSGGTAALGCLVKDYFPEPVTVSWNSGALTSGVHTFPAVLQ
SSGLYSLSSVVTVPSSSLGTQTYICNVNHKPSNTKVDKKVEPKSC

### Construct #3

### EPCAM VH CH1-CD3 CROSSFAB VH-CL-Fc (Knob) P329GLA-LA

Signaling peptide in bold, italics underlined, then 3 amino acids-AAS due to cloning sites; Cloning sites Nhe I GCTAGC and Nsi I ATGCAT are underlined, larger font

EPCAM-VH-in bold; CH1-underlined; 2xG4S linker; CD3 VH bold italics; CL in italics underlined; IgG Fc chain with LA-LA, (L234 and L235 changed to A) mutations shown in bold, underlined, and P329G mutation, P changed to G, bold underlined.

The knob mutations in Fc domain were S354C and T366W shown in bold larger font, italics.

After EPCAM VH, AS amino acids can be included before start of CH1.

### Nucleotide Sequence

(SEQ ID NO: 9)
ATGGAGACAGACACACTCCTGCTATGGGTACTGCTGCTCTGGGTTCCAGGTTCCACTGGCGCCGC
TAGCCAGGTGCAGCTGGTGCAGAGCGGCAGCGAACTGAAAAAACCGGGCGCGA
GCGTGAAAGTGAGCTGCAAAGCGAGCGGCTATACCTTTACCAACTATGGCATG
AACTGGGTGCGCCAGGCGCCGGGCCAGGGCCTGGAATGGATGGGCTGGATTA
ACACCTATACCGGCGAACCGACCTATGCGGATGATTTTAAAGGCCGCTTTGTGT
TTAGCCTGGATACCAGCGTGAGCACCGCGTATCTGCAGATTAGCAGCCTGAAA
GCGGAAGATACCGCGGTGTATTATTGCGCGCGCTGGCTGCGCGATTTTGATTA
TTGGGGCGCGGGCACCACCGTGACCGTGAGCAGCACA AAA GGT CCT TCC GTA
TTT CCG CTC GCA CCC AGC TCT AAG TCA ACC TCT GGC GGT ACT GCA GCC
CTG GGT TGC CTC GTA AAG GAC TAT TTT CCT GAG CCA GTA ACA GTT TCT
TGG AAC AGC GGG GCA CTT ACG AGC GGT GTT CAT ACG TTC CCT GCA GTG
TTG CAA TCC AGC GGC CTT TAT TCA TTG TCT TCA GTT GTA ACG GTT CCT
TCT AGT AGT TTG GGG ACC CAG ACA TAT ATC TGC AAC GTG AAC CAT AAG
CCA AGC AAT ACC AAA GTT GAT AAG AAG GTC GAA CCT AAG TCC TGC GAC
GGC GGG GGA GGA TCT GGC GGG GGA GGC AGT GAG GTA CAA TTG TTG GAG
TCA GGC GGC GGT CTT GTC CAA CCG GGA GGG TCC CTG AGA CTG TCC TGT
GCG GCC AGC GGG TTT ACT TTT TCA ACA TAT GCC ATG AAC TGG GTT CGG
CAA GCA CCA GGT AAG GGA CTG GAA TGG GTT AGT CGA ATT AGG TCC AAG
TAT AAT AAT TAC GCA ACC TAC TAC GCT GAC TCT GTC AAG GGG CGG TTT
ACC ATA TCT AGG GAT GAC TCC AAA AAC ACA TTG TAC TTG CAA ATG AAC
AGC CTG AGG GCA GAA GAC ACC GCA GTC TAC TAT TGT GTA CGC CAC GGA
AAC TTC GGG AAT AGT TAT GTC TCC TGG TTC GCT TAC TGG GGT CAG GGA
ACA CTG GTC ACA GTC TCA TCA GCC AGT GTA GCG GCC CCG TCC GTT TTC
ATA TTC CCT CCT TCC GAC GAG CAG TTG AAA AGC GGT ACG GCG AGC GTT
GTG TGC TTG TTG AAC AAC TTC TAC CCA CGC GAA GCC AAG GTC CAA TGG
AAG GTA GAC AAC GCA CTG CAG AGT GGT AAC TCA CAG GAA TCA GTG ACG
GAA CAG GAC TCA AAA GAT AGT ACT TAC AGT CTT TCT TCC ACA CTG ACA
CTC AGT AAG GCC GAT TAT GAG AAA CAT AAA GTA TAC GCA TGT GAA GTA
ACT CAC CAG GGT CTC AGT TCA CCA GTA ACT AAG TCT TTC AAT CGC GGG
GAA TGC GAC AAA ACA CAC ACC TGT CCC CCC TGT CCA GCC CCA GAG GCA
GCT GGC GGC CCT AGT GTG TTC TTG TTC CCG CCC AAG CCA AAA GAT ACA
CTG ATG ATT AGC CGG ACC CCT GAG GTA ACT TGT GTG GTG GTG GAC GTG
TCT CAT GAG GAC CCA GAG GTA AAA TTC AAC TGG TAC GTA GAC GGC GTC
GAG GTC CAT AAT GCC AAA ACC AAG CCA CGG GAG GAG CAG TAT AAT TCC
ACT TAT CGC GTA GTC TCT GTA CTT ACA GTT CTT CAC CAA GAT TGG TTG
AAC GGA AAA GAA TAC AAG TGT AAA GTT AGC AAT AAG GCG CTC GGA GCT
CCG ATC GAA AAA ACA ATC TCC AAA GCA AAA GGG CAA CCC CGA GAA CCA
CAG GTA TAC ACC CTG CCG CCG TGC CGA GAC GAG CTG ACG AAA AAC CAA
GTG TCC CTG TGG TGC TTG GTG AAG GGC TTT TAT CCA AGT GAC ATT GCA
GTT GAA TGG GAG TCT AAC GGA CAG CCT GAA AAT AAC TAT AAG ACC ACG
CCA CCA GTC CTT GAT AGC GAT GGA TCT TTT TTT CTC TAT AGC AAG TTG
ACT GTA GAT AAA TCA CGA TGG CAA CAA GGC AAT GTC TTT TCA TGC AGC
GTT
ATGCATGAGGCTCTGCACAACCACTACACGCAGAAGAGCCTCTCCCTGTCTCCCGGGAAATGA.

### Amino Acid Sequence of EPCAM VH, SEQ ID NO: 10

QVQLVQSGSELKKPGASVKVSCKASGYTFTNYGMNWVRQAPGQGLEWMGW
INTYTGEPTYADDFKGRFVFSLDTSVSTAYLQISSLKAEDTA VYYCARW
LRDFDYWGAGTTVTVSS
CD3 VH,
SEQ ID NO: 11
EVQLLESGGGLVQPGGSLRLSCAASGFTFSTYAMNWVRQAPGKGLEWVSR
IRSKYNNYATYYADSVKGRFTISRDDSKNTLYLQMNSLRAEDTAVYYCVR
HGNFGNSYVSWFAYWGQGTLVTVSS

### Amino Acid Sequence of Construct #3 (Not Including Signaling Peptide)

(SEQ ID NO: 12)
QVQLVQSGSELKKPGASVKVSCKASGYTFTNYGMNWVRQAPGQGLEWMGW
INTYTGEPTYADDFKGRFVFSLDTSVSTAYLQISSLKAEDTAVYYCARWL
RDFDYWGAGTTVTVSSTKGPSVFPLAPSSKSTSGGTAALGCLVKDYFPEP
VTVSWNSGALTSGVHTFPAVLOSSGLYSLSSVVTVPSSSLGTQTYICNVN
HKPSNTKVDKKVEPKSCDGGGGSGGGGSEVQLLESGGGLVQPGGSLRLSC
AASGFTFSTYAMNWVRQAPGKGLEWVSRIRSKYNNYATYYADSVKGRFTI
SRDDSKNTLYLQMNSLRAEDTAVYYCVRHGNFGNSYVSWFAYWGQGTLVT
VSSASVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSP
VTKSFNRGECDKTHTCPPCPAPEAAGGPSVFLFPPKPKDTLMISRTPEVT
CVVVDVSHEDPEVKFNWYVDGVEVHNAKTKPREEQYNSTYRVVSVLTVLH
QDWLNGKEYKCKVSNKAL [symbol] APIEKTISKAKGQPREPQVYTLPP [symbol] RD
ELTKNQVSL [symbol] CLVKGFYPSDIAVEWESNGQPENNYKTTPPVLDSDGSFF
LYSKLTVDKSRWQQGNVFSCSVMHEALHNHYTQKSLSLSPGK

### Construct #4 EPCAM VH-CH1-Fc (Hole) P329GLA-LA

Construct #4 used the same P329G and LALA mutations as in Construct #3, shown in bold. The hole mutations were Y349C, T366S, L368A and Y407V shown in bold, larger fond, italics. Cloning sites Nhe I GCTAGC and Nsi I ATGCAT are underlined

Signaling peptide underlined, bold, italics, then 9 nucleotides encoding 3 amino-acids AAS (cloning sites), regular font, italics; EPCAM-VH-bold, CH1 underlined, then Fc with P329GLA-LA and hole mutations

### Nucleotide

(SEQ ID NO: 13)
ATGGAGACAGACACACTCCTGCTATGGGTACTGCTGCTCTGGGTTCCAGG
TTCCACTGGCGCCGCTAGCCAGGTGCAGCTGGTGCAGAGCGGCAGCGAAC
TGAAAAAACCGGGCGCGAGCGTGAAAGTGAGCTGCAAAGCGAGCGGCTAT
ACCTTTACCAACTATGGCATGAACTGGGTGCGCCAGGCGCCGGGCCAGGG
CCTGGAATGGATGGGCTGGATTAACACCTATACCGGCGAACCGACCTATG
CGGATGATTTTAAAGGCCGCTTTGTGTTTAGCCTGGATACCAGCGTGAGC
ACCGCGTATCTGCAGATTAGCAGCCTGAAAGCGGAAGATACCGCGGTGTA
TTATTGCGCGCGCTGGCTGCGCGATTTTGATTATTGGGGCGCGGGCACCA
CCGTGACCGTGAGCAGCACC AAA GGT CCA AGC GTA TTT CCA
CTC GCA CCG TCC TCC AAA TCA ACG AGT GGA GGT ACT
GCG GCG TTG GGA TGC TTG GTG AAAGAC TAT TTC CCA GAG
CCG GTG ACA GTT AGT TGG AAC TCA GGC GCG CTG ACT AG
T GGT GTT CAC ACA TTT CCT GCA GTT TTG CAATCC TCA G
GC CTC TAT AGC CTG TCA AGC GTC GTG ACA GTA CCT AGC
AGC TCA CTT GGC ACG CAG ACA TAT ATT TGT AAT GTC
AACCAT AAG CCC TCT AAC ACA AAG GTA GAT AAG AAG GTT
GAA CCC AAG TCC TGT GAC AAA ACG CAC ACT TGT CCA CC
A TGT CCA GCGCCC GAA [symbol] GGG GGC CCA AGC
GTG TTC CTC TTC CCT CCC AAG CCA AAA GAC ACC CTT AT
G ATC TCA AGG ACT CCA GAA GTGACA TGC GTA GTC GTT G
AC GTA AGT CAC GAG GAT CCG GAA GTG AAG TTC AAC TGG
TAC GTG GAC GGT GTG GAG GTA CAT AAC GCGAAG ACT AAG
CCC AGA GAA GAA CAA TAT AAC TCA ACC TAC CGG GTC GT
T TCT GTG CTC ACA GTG CTC CAC CAG GAC TGG CTT AACG
GA AAA GAG TAT AAA TGC AAA GTA TCT AAT AAA GCG CTC [symbol]
GCG CCC ATA GAG AAA ACT ATT TCT AAA GCA AAA G
GT CAA CCACGG GAG CCG CAG GTT [symbol] ACA CTT CCA CCG
TCC AGG GAT GAA CTT ACT AAG AAC CAG GTA TCT CTT [symbol]
TGT [symbol] GTG AAA GGTTTT TAT CCT AGT GAC ATC
GCT GTC GAG TGG GAG AGC AAC GGT CAG CCG GAG AAT AA
C TAT AAG ACC ACA CCT CCG GTT CTG GATTCT GAC GGC
TCT TTC TTC CTG [symbol] TCT AAG CTT ACA GTC GAT AAA
AGT CGA TGG CAA CAA GGG AAT GTT TTT AGC TGC TCT GT
GATGCATGAGGCTCTGCACAACCACTACACGCAGAAGAGCCTCTCCCTGT
CTCCCGGGAAATGA

### Amino Acid of Construct #4 (Not Including Signaling Peptide)

(SEQ ID NO: 14)
QVQLVQSGSELKKPGASVKVSCKASGYTFTNYGMNWVRQAPGQGLEWMGW
INTYTGEPTYADDFKGRFVFSLDTSVSTAYLQISSLKAEDTAVYYCARWL
RDFDYWGAGTTVTVSSTKGPSVFPLAPSSKSTSGGTAALGCLVKDYFPEP
VTVSWNSGALTSGVHTFPAVLQSSGLYSLSSVVTVPSSSLGTQTYICNVN
HKPSNTKVDKKVEPKSCDKTHTCPPCPAPEAAGGPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALGAPIEKTISKAKGQPREPQV [symbol] TL
PPSRDELTKNQVSL [symbol] C [symbol] VKGFYPSDIAVEWESNGQPENNYKTTPP
VLDSDGSFFL [symbol] SKLTVDKSRWQQGNVFSCSVMHEALHNHYTQKSLSLS
PGK

### Example 3. The Sequence of EPCAM-CD3 Antibody (FIG. 1B Structure)

FIG. 1B shows the structure of humanized EPCAM ScFv-linker-CD3 ScFv-His tag (BITE) consisting of one DNA construct (PBM0065). Signaling peptide underlined

Nucleotide sequence, PBM0065:

(SEQ ID NO: 15)
ATGGAGACAGACACACTCCTGCTATGGGTACTGCTGCTCTGGGTTCCAGG
TTCCACTGGCGCCGCTAGCCAGGTGCAACTCGTACAAAGCGGTTCCGAAC
TGAAGAAACCTGGTGCGAGCGTAAAGGTGTCCTGTAAAGCATCTGGATAC
ACCTTCACCAACTACGGAATGAACTGGGTTCGACAGGCACCGGGCCAAGG
CTTGGAGTGGATGGGGTGGATCAACACCTACACGGGTGAACCTACGTACG
CAGATGATTTCAAAGGTCGCTTCGTGTTTTCCCTGGATACTTCTGTGTCC
ACAGCGTACCTCCAGATTTCCAGTCTGAAGGCCGAGGACACAGCCGTATA
TTACTGTGCTCGGTGGTTGCGCGATTTTGATTATTGGGGCGCAGGTACTA
CTGTGACAGTAAGCTCCGGCGGAGGCGGGTCAGGGGGCGGTGGATCCGGC
GGAGGGGGATCAGAAATCGTGCTCACTCAATCACCAGCGACCTTGAGCTT
GTCACCTGGGGAGCGGGCGACGCTGTCCTGTAGTGCGTCATCATCTATTT
CATATATGCACTGGTATCAACAGAAGCCAGGACAGGCGCCTCGCCTGTTG
ATTTACGATACTAGCAAATTGGCAACGGGGATACCCGCTCGATTCAGCGG
CAGCGGATCTGGGACGGATTTCACATTGACCATTTCTAGCCTTGAACCAG
AGGACTTCGCAGTATATTACTGCCATCAGAGGTCCAGCTATCCATACACA
TTTGGCGGAGGGACGAAATTGGAAATCAAGGGGGGAGGCGGGTCTGGGGG
AGGCGGAAGTGGGGGAGGAGGAAGCGAAGTTCAGCTGCTCGAATCCGGCG
GCGGCCTTGTTCAGCCAGGTGGTAGCTTGAGGCTCAGTTGTGCTGCATCT
GGGTTTACATTCTCAACTTATGCGATGAACTGGGTGAGGCAAGCACCTGG
AAAGGGACTTGAGTGGGTCTCAAGAATTCGCTCCAAATACAACAACTATG
CGACGTATTACGCAGACTCAGTGAAAGGACGGTTTACGATATCACGGGAC
GATTCAAAGAATACACTGTATTTGCAGATGAATTCTCTTAGGGCCGAAGA
CACTGCCGTATACTATTGTGTACGCCACGGTAATTTTGGCAATAGCTATG
TATCTTGGTTCGCGTACTGGGGCCAAGGCACCCTTGTTACTGTGTCTAGT
GGGGGCGGGGGGAGTGGTGGCGGAGGAAGTGGCGGGGGGGGATCTCAAGC
GGTGGTTACTCAAGAGCCCTCCCTTACTGTTTCTCCGGGCGGGACGGTCA
CCTTGACTTGTGGCAGTTCAACAGGGGCAGTCACTACTAGTAATTATGCG
AATTGGGTCCAAGAAAAGCCGGGCCAAGCTTTCCGGGGACTCATCGGAGG
AACAAATAAAAGGGCACCCGGCACACCCGCGCGCTTTTCCGGGAGTCTTC
TGGGCGGCAAGGCAGCCCTCACTCTCTCTGGGGCTCAACCTGAGGACGAG
GCTGAGTACTATTGTGCCCTCTGGTACTCAAACCTGTGGGTCTTTGGAGG
GGGAACCAAGCTTACGGTCTTGCATCATCACCATCATCATTAATGA

Amino acid sequence

Signaling peptide underlined.

EPCAM ScFv (VH-G4Sx3-VL): VH bold, VL bold and underlined.

CD3 ScFv (VH-G4Sx3-VL): VH italics, VL-italics and underlined, then 6xHis tag.

(SEQ ID NO: 16)
METDTLLLWVLLLWVPGSTGAASQVQLVQSGSELKKPGASVKVSCKASGY
TFTNYGMNWVRQAPGQGLEWMGWINTYTGEPTYADDFKGRFVFSLDTSVS
TAYLQISSLKAEDTAVYYCARWLRDFDYWGAGTTVTVSSGGGGSGGGGSG
GGGSEIVLTQSPATLSLSPGERATLSCSASSSISYMHWYQQKPGQAPRLL
IYDTSKLATGIPARFSGSGSGTDFTLTISSLEPEDFAVYYCHQRSSYPYT
FGGGTKLEIKGGGGSGGGGSGGGGSEVQLLESGGGLVQPGGSLRLSCAAS
GFTFSTYAMNWVRQAPGKGLEWVSRIRSKYNNYATYYADSVKGRFTISRD
DSKNTLYLQMNSLRAEDTAVYYCVRHGNFGNSYVSWFAYWGQGTLVTVSS
GGGGSGGGGSGGGGSQAVVTQEPSLTVSPGGTVTLTCGSSTGAVTTSNYA
NWVQEKPGQAFRGLIGGTNKRAPGTPARFSGSLLGGKAALTLSGAQPEDE
AEYYCALWYSNLWVFGGGTKLTVLHHHHHH

### Example 4. The Sequence of EPCAM-CD3 Antibody (FIG. 1C Structure)

FIG. 1C shows the structure of humanized EPCAM ScFv-linkerG4Sx3-CD3 ScFv-human Fc, which consists of one DNA construct (PBM0070).

DNA (PMC1367) was cloned to the same sites as in Example 2 to pYD11 vector.

Nucleotide sequence: signaling peptide in bold, underlined, humanized EpCAM scFv-G4Sx3 CD3 ScFv (BITE SEQ PBM0065 without 6xHis tag); human Fc underlined:

(SEQ ID NO: 17)
ATGGAGACAGACACACTCCTGCTATGGGTACTGCTGCTCTGGGTTCCAGG
TTCCACTGGCGCCGCTAGCCAGGTGCAACTCGTACAAAGCGGTTCCGAAC
TGAAGAAACCTGGTGCGAGCGTAAAGGTGTCCTGTAAAGCATCTGGATAC
ACCTTCACCAACTACGGAATGAACTGGGTTCGACAGGCACCGGGCCAAGG
CTTGGAGTGGATGGGGTGGATCAACACCTACACGGGTGAACCTACGTACG
CAGATGATTTCAAAGGTCGCTTCGTGTTTTCCCTGGATACTTCTGTGTCC
ACAGCGTACCTCCAGATTTCCAGTCTGAAGGCCGAGGACACAGCCGTATA
TTACTGTGCTCGGTGGTTGCGCGATTTTGATTATTGGGGCGCAGGTACTA
CTGTGACAGTAAGCTCCGGCGGAGGCGGGTCAGGGGGCGGTGGATCCGGC
GGAGGGGGATCAGAAATCGTGCTCACTCAATCACCAGCGACCTTGAGCTT
GTCACCTGGGGAGCGGGCGACGCTGTCCTGTAGTGCGTCATCATCTATTT
CATATATGCACTGGTATCAACAGAAGCCAGGACAGGCGCCTCGCCTGTTG
ATTTACGATACTAGCAAATTGGCAACGGGGATACCCGCTCGATTCAGCGG
CAGCGGATCTGGGACGGATTTCACATTGACCATTTCTAGCCTTGAACCAG
AGGACTTCGCAGTATATTACTGCCATCAGAGGTCCAGCTATCCATACACA
TTTGGCGGAGGGACGAAATTGGAAATCAAGGGGGGAGGCGGGTCTGGGGG
AGGCGGAAGTGGGGGAGGAGGAAGCGAAGTTCAGCTGCTCGAATCCGGCG
GCGGCCTTGTTCAGCCAGGTGGTAGCTTGAGGCTCAGTTGTGCTGCATCT
GGGTTTACATTCTCAACTTATGCGATGAACTGGGTGAGGCAAGCACCTGG
AAAGGGACTTGAGTGGGTCTCAAGAATTCGCTCCAAATACAACAACTATG
CGACGTATTACGCAGACTCAGTGAAAGGACGGTTTACGATATCACGGGAC
GATTCAAAGAATACACTGTATTTGCAGATGAATTCTCTTAGGGCCGAAGA
CACTGCCGTATACTATTGTGTACGCCACGGTAATTTTGGCAATAGCTATG
TATCTTGGTTCGCGTACTGGGGCCAAGGCACCCTTGTTACTGTGTCTAGT
GGGGGCGGGGGGAGTGGTGGCGGAGGAAGTGGCGGGGGGGGATCTCAAGC
GGTGGTTACTCAAGAGCCCTCCCTTACTGTTTCTCCGGGCGGGACGGTCA
CCTTGACTTGTGGCAGTTCAACAGGGGCAGTCACTACTAGTAATTATGCG
AATTGGGTCCAAGAAAAGCCGGGCCAAGCTTTCCGGGGACTCATCGGAGG
AACAAATAAAAGGGCACCCGGCACACCCGCGCGCTTTTCCGGGAGTCTTC
TGGGCGGCAAGGCAGCCCTCACTCTCTCTGGGGCTCAACCTGAGGACGAG
GCTGAGTACTATTGTGCCCTCTGGTACTCAAACCTGTGGGTCTTTGGAGG
GGGAACCAAGCTTACGGTCTTGTCTAGAGAAAACCTGTATTTTCAGGGCA
CTCACACATGCCCACCGTGCCCAGCACCTGAACTCCTGGGGGGACCGTCA
GTCTTCCTCTTCCCCCCAAAACCCAAGGACACCCTCATGATCTCCCGGAC
CCCTGAGGTCACATGCGTGGTGGTGGACGTGAGCCACGAAGACCCTGAGG
TCAAGTTCAACTGGTACGTGGACGGCGTGGAGGTGCATAATGCCAAGACA
AAGCCGCGGGAGGAGCAGTACAACAGCACGTACCGTGTGGTCAGCGTCCT
CACCGTCCTGCACCAGGACTGGCTGAATGGCAAGGAGTACAAGTGCAAGG
TCTCCAACAAAGCCCTCCCAGCCCCCATCGAGAAAACCATCTCCAAAGCC
AAAGGGCAGCCCCGAGAACCACAGGTGTACACCCTGCCCCCATCCCGGGA
TGAGCTGACCAAGAACCAGGTCAGCCTGACCTGCCTGGTCAAAGGCTTCT
ATCCCAGCGACATCGCCGTGGAGTGGGAGAGCAATGGGCAGCCGGAGAAC
AACTACAAGACCACGCCTCCCGTGCTGGACTCCGACGGCTCCTTCTTCCT
CTACAGCAAGCTCACCGTGGACAAGAGCAGGTGGCAGCAGGGGAACGTCT
TCTCATGCTCCGTGATGCATGAGGCTCTGCACAACCACTACACGCAGAAG
AGCCTCTCCCTGTCTCCCGGGAAATGA

Amino acid sequence:

Signaling peptide, bold underlined;

| (SEQ ID NO: 18) | |
|---|---|
| | METDTLLLWVLLLWVPGSTGAAS |

Amino acid of EPCAMscFv-G4Sx3-CD3ScFv (BITE, in bold)—human Fc domain (underlined) is shown below.

(SEQ ID NO: 19)
QVQLVQSGSELKKPGASVKVSCKASGYTFTNYGMNWVRQAPGQGLEWMGW
INTYTGEPTYADDFKGRFVFSLDTSVSTAYLQISSLKAEDTAVYYCARWL
RDFDYWGAGTTVTVSSGGGGSGGGGSGGGGSEIVLTQSPATLSLSPGERA
TLSCSASSSISYMHWYQQKPGQAPRLLIYDTSKLATGIPARFSGSGSGTD
FTLTISSLEPEDFAVYYCHQRSSYPYTFGGGTKLEIKGGGGSGGGGSGGG
GSEVQLLESGGGLVQPGGSLRLSCAASGFTFSTYAMNWVRQAPGKGLEWV
SRIRSKYNNYATYYADSVKGRFTISRDDSKNTLYLQMNSLRAEDTAVYYC
VRHGNFGNSYVSWFAYWGQGTLVTVSSGGGGSGGGGSGGGGSQAVVTQEP
SLTVSPGGTVTLTCGSSTGAVTTSNYANWVQEKPGQAFRGLIGGTNKRAP
GTPARFSGSLLGGKAALTLSGAQPEDEAEYYCALWYSNLWVFGGGTKLTV
LSRENLYFQGTHTCPPCPAPELLGGPSVFLFPPKPKDTLMISRTPEVTCV
VVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTV
DKSRWQQGNVFSCSVMHEALHNHYTQKSLSLSPGK

The amino acid sequence of CH2-CH3 of human IgG1 Fc is shown below.

(SEQ ID NO: 20)
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHE
ALHNHYTQKSLSLSPGK

### Example 5. The Sequence of DNA Template of EPCAM-CD3 Antibody to Transcribe EpCAM-CD3 Ab RNA (FIG. 1C Structure)

The nucleotide sequence PMC1367 (template to make FIG. 1C structure) is inserted into DNA which is a template for in vitro RNA transcription to generate PMC1396. The structure is SP6 promoter: 5′-ATTTAGGTGACACTATAG-3′ (underlined below, SEQ ID NO: 21); 5′UTR (italics), EpCAM-CD3-human Fc sequence in bold and starting with ATG start codon (underlined) and ending with stop codon TGA (underlined); 3′UTR (italics); 110 amino-acids poly A tail with linker in the middle).

Before in vitro transcription reaction, the sequence is linearized with BglII enzyme, AGATCT, underlined at the end of poly A tail or with Asc I site GGCGCGCC (Italics, bold), 6 bases 3′ after poly A tail.

During or after in vitro transcription reaction, the 5′Cap is added for increased stability of RNA. The RNA is checked on agarose gel and then used to transfect 293 cells to generate EPCAM-CD3-hFc antibody.

(SEQ ID NO: 22)
ATTTAGGTGACACTATAGAATACAAGCTTacatttgcttctgacacaactgtgttcact
agcaacctcaaacagacaccatgGAGACAGACACACTCCTGCTATGGGTACTGCTGCTC
TGGGTTCCAGGTTCCACTGGCGCCGCTAGCCAGGTGCAACTCGTACAAAGCGGTTCCGA
ACTGAAGAAACCTGGTGCGAGCGTAAAGGTGTCCTGTAAAGCATCTGGATACACCTTCA
CCAACTACGGAATGAACTGGGTTCGACAGGCACCGGGCCAAGGCTTGGAGTGG
ATGGGGTGGATCAACACCTACACGGGTGAACCTACGTACGCAGATGATTTCAA
AGGTCGCTTCGTGTTTTCCCTGGATACTTCTGTGTCCACAGCGTACCTCCAGAT
TTCCAGTCTGAAGGCCGAGGACACAGCCGTATATTACTGTGCTCGGTGGTTGC
GCGATTTTGATTATTGGGGCGCAGGTACTACTGTGACAGTAAGCTCCGGCGGA
GGCGGGTCAGGGGGCGGTGGATCCGGCGGAGGGGGATCAGAAATCGTGCTCA
CTCAATCACCAGCGACCTTGAGCTTGTCACCTGGGGAGCGGGCGACGCTGTCC
TGTAGTGCGTCATCATCTATTTCATATATGCACTGGTATCAACAGAAGCCAGGA
CAGGCGCCTCGCCTGTTGATTTACGATACTAGCAAATTGGCAACGGGGATACC
CGCTCGATTCAGCGGCAGCGGATCTGGGACGGATTTCACATTGACCATTTCTA
GCCTTGAACCAGAGGACTTCGCAGTATATTACTGCCATCAGAGGTCCAGCTATC
CATACACATTTGGCGGAGGGACGAAATTGGAAATCAAGGGGGGAGGCGGGTCT
GGGGGAGGCGGAAGTGGGGGAGGAGGAAGCGAAGTTCAGCTGCTCGAATCCG
GCGGCGGCCTTGTTCAGCCAGGTGGTAGCTTGAGGCTCAGTTGTGCTGCATCT
GGGTTTACATTCTCAACTTATGCGATGAACTGGGTGAGGCAAGCACCTGGAAA
GGGACTTGAGTGGGTCTCAAGAATTCGCTCCAAATACAACAACTATGCGACGT
ATTACGCAGACTCAGTGAAAGGACGGTTTACGATATCACGGGACGATTCAAAG
AATACACTGTATTTGCAGATGAATTCTCTTAGGGCCGAAGACACTGCCGTATAC
TATTGTGTACGCCACGGTAATTTTGGCAATAGCTATGTATCTTGGTTCGCGTAC
TGGGGCCAAGGCACCCTTGTTACTGTGTCTAGTGGGGGCGGGGGGAGTGGTG
GCGGAGGAAGTGGCGGGGGGGGATCTCAAGCGGTGGTTACTCAAGAGCCCTC
CCTTACTGTTTCTCCGGGCGGGACGGTCACCTTGACTTGTGGCAGTTCAACAG
GGGCAGTCACTACTAGTAATTATGCGAATTGGGTCCAAGAAAAGCCGGGCCAA
GCTTTCCGGGGACTCATCGGAGGAACAAATAAAAGGGCACCCGGCACACCCGC
GCGCTTTTCCGGGAGTCTTCTGGGCGGCAAGGCAGCCCTCACTCTCTCTGGGG
CTCAACCTGAGGACGAGGCTGAGTACTATTGTGCCCTCTGGTACTCAAACCTGT
GGGTCTTTGGAGGGGGAACCAAGCTTACGGTCTTGTCTAGAGAAAACCTGTATTTTCAG
GGCACTCACACATGCCCACCGTGCCCAGCACCTGAACTCCTGGGGGGACCGTCAGTCTT
CCTCTTCCCCCCAAAACCCAAGGACACCCTCATGATCTCCCGGACCCCTGAGGTCACA
TGCGTGGTGGTGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAACAGCACGTA
CCGTGTGGTCAGCGTCCTCACCGTCCTGCACCAGGACTGGCTGAATGGCAAGGAGTACA
AGTGCAAGGTCTCCAACAAAGCCCTCCCAGCCCCCATCGAGAAAACCATCTCCAAAGCC
AAAGGGCAGCCCCGAGAACCACAGGTGTACACCCTGCCCCCATCCCGGGATGAGCTGAC
CAAGAACCAGGTCAGCCTGACCTGCCTGGTCAAAGGCTTCTATCCCAGCGACATCGCCG
TGGAGTGGGAGAGCAATGGGCAGCCGGAGAACAACTACAAGACCACGCCTCCCGTGCTG
GACTCCGACGGCTCCTTCTTCCTCTACAGCAAGCTCACCGTGGACAAGAGCAGGTGGCA
GCAGGGGAACGTCTTCTCATGCTCCGTGATGCATGAGGCTCTGCACAACCACTACACGC
AGAAGAGCCTCTCCCTGTCTCCCGGGAAATGAgctcgctttcttgctgtccaatttcta
ttaaaggttcctttgttccctaagtccaactactaaactgggggatattatgaagggcc
ttgagcatctggattctgcctaataaaaaacatttattttcattgcagctcgctttctt
gctgtccaatttctattaaaggttcctttgttccctaagtccaactactaaactggggg
atattatgaagggccttgagcatctggattctgcctaataaaaaacatttattttcatt
gcaGTCGACTCTAGAAAAAAAAAAAAAAAAAAAAAAAAAAAAAAAAAAAAAAAAAGGAT
CCCCGGGCGAGCTCCCAAAAAAAAAAAAAAAAAAAAAAAAAAAAAACCGAATTCCTGCA
GCTCGAGAAAAAAAAAAAAAAAAAAAAAAAAAAAAAAAAAAAAAAAAAGATCTGGCGCG
CCGTAATCATGTCATAGCTGTTTCCTGTGTGAAATTGTTATCCGCTCACAATTCCACAC
AACATACGAGCCGGAAGCATAAAGTGTAAAGCCTGGGGTGCCTAATGAGTGAGCTAACT
CACATTAATTGCGTTGCGCTCACTGCCCGCTTTCCAGTCGGGAAACCTGTCGTGCCAGC
TGCATTAATGAATCGGCCAACGCGCGGGGAGAGGCGGTTTGCGTATTGGGCGCTCTTCC
GCTTCCTCGCTCACTGACTCGCTGCGCTCGGTCGTTCGGCTGCGGCGAGCGGTATCAGC
TCACTCAAAGGCGGTAATACGGTTATCCACAGAATCAGGGGATAACGCAGGAAAGAACA
TGTGAGCAAAAGGCCAGCAAAAGGCCAGGAACCGTAAAAAGGCCGCGTTGCTGGCGTTT
TTCCATAGGCTCCGCCCCCCTGACGAGCATCACAAAAATCGACGCTCAAGTCAGAGGTG
GCGAAACCCGACAGGACTATAAAGATACCAGGCGTTTCCCCCTGGAAGCTCCCTCGTGC
GCTCTCCTGTTCCGACCCTGCCGCTTACCGGATACCTGTCCGCCTTTCTCCCTTCGGGA
AGCGTGGCGCTTTCTCATAGCTCACGCTGTAGGTATCTCAGTTCGGTGTAGGTCGTTCG
CTCCAAGCTGGGCTGTGTGCACGAACCCCCCGTTCAGCCCGACCGCTGCGCCTTATCCG
GTAACTATCGTCTTGAGTCCAACCCGGTAAGACACGACTTATCGCCACTGGCAGCAGCC
ACTGGTAACAGGATTAGCAGAGCGAGGTATGTAGGCGGTGCTACAGAGTTCTTGAAGTG
GTGGCCTAACTACGGCTACACTAGAAGAACAGTATTTGGTATCTGCGCTCTGCTGAAGC
CAGTTACCTTCGGAAAAAGAGTTGGTAGCTCTTGATCCGGCAAACAAACCACCGCTGGT
AGCGGTGGTTTTTTTGTTTGCAAGCAGCAGATTACGCGCAGAAAAAAAGGATCTCAAGA
AGATCCTTTGATCTTTTCTACGGGGTCTGACGCTCAGTGGAACGAAAACTCACGTTAAG
GGATTTTGGTCATGAGATTATCAAAAAGGATCTTCACCTAGATCCTTTTAAATTAAAAA
TGAAGTTTTAAATCAATCTAAAGTATATATGAGTAAACTTGGTCTGACAGTTACCAATG
CTTAATCAGTGAGGCACCTATCTCAGCGATCTGTCTATTTCGTTCATCCATAGTTGCCT
GACTCCCCGTCGTGTAGATAACTACGATACGGGAGGGCTTACCATCTGGCCCCAGTGCT
GCAATGATACCGCGAGACCCACGCTCACCGGCTCCAGATTTATCAGCAATAAACCAGCC
AGCCGGAAGGGCCGAGCGCAGAAGTGGTCCTGCAACTTTATCCGCCTCCATCCAGTCTA
TTAATTGTTGCCGGGAAGCTAGAGTAAGTAGTTCGCCAGTTAATAGTTTGCGCAACGTT
GTTGCCATTGCTACAGGCATCGTGGTGTCACGCTCGTCGTTTGGTATGGCTTCATTCAG
CTCCGGTTCCCAACGATCAAGGCGAGTTACATGATCCCCCATGTTGTGCAAAAAAGCGG
TTAGCTCCTTCGGTCCTCCGATCGTTGTCAGAAGTAAGTTGGCCGCAGTGTTATCACTC
ATGGTTATGGCAGCACTGCATAATTCTCTTACTGTCATGCCATCCGTAAGATGCTTTTC
TGTGACTGGTGAGTACTCAACCAAGTCATTCTGAGAATAGTGTATGCGGCGACCGAGTT
GCTCTTGCCCGGCGTCAATACGGGATAATACCGCGCCACATAGCAGAACTTTAAAAGTG
CTCATCATTGGAAAACGTTCTTCGGGGCGAAAACTCTCAAGGATCTTACCGCTGTTGAG
ATCCAGTTCGATGTAACCCACTCGTGCACCCAACTGATCTTCAGCATCTTTTACTTTCA
CCAGCGTTTCTGGGTGAGCAAAAACAGGAAGGCAAAATGCCGCAAAAAAGGGAATAAGG
GCGACACGGAAATGTTGAATACTCATACTCTTCCTTTTTCAATATTATTGAAGCATTTA
TCAGGGTTATTGTCTCATGAGCGGATACATATTTGAATGTATTTAGAAAAATAAACAAA
TAGGGGTTCCGCGCACATTTCCCCGAAAAGTGCCACCTGACGTCTAAGAAACCATTATT
ATCATGACATTAACCTATAAAAATAGGCGTATCACGAGGCCCTTTCGTCTCGCGCGTTT
CGGTGATGACGGTGAAAACCTCTGACACATGCAGCTCCCGGAGACGGTCACAGCTTGTC
TGTAAGCGGATGCCGGGAGCAGACAAGCCCGTCAGGGCGCGTCAGCGGGTGTTGGCGGG
TGTCGGGGCTGGCTTAACTATGCGGCATCAGAGCAGATTGTACTGAGAGTGCACCATTC
GACGCTCTCCCTTATGCGACTCCTGCATTAGGAAGCAGCCCAGTAGTAGGTTGAGGCCG
TTGAGCACCGCCGCCGCAAGGAATGGTGCATGCAAGGAGATGGCGCCCAACAGTCCCCC
GGCCACGGGGCCTGCCACCATACCCACGCCGAAACAAGCGCTCATGAGCCCGAAGTGGC
GAGCCCGATCTTCCCCATCGGTGATGTCGGCGATATAGGCGCCAGCAACCGCACCTGTG
GCGCCGGTGATGCCGGCCACGATGCGTCCGGCGTAGAGGATCTGGCTAGCGATGACCCT
GCTGATTGGTTCGCTGACCATTTCCGGGTGCGGGACGGCGTTACCAGAAACTCAGAAGG
TTCGTCCAACCAAACCGACTCTGACGGCAGTTTACGAGAGAGATGATAGGGTCTGCTTC
AGTAAGCCAGATGCTACACAATTAGGCTTGTACATATTGTCGTTAGAACGCGGCTACAA
TTAATACATAACCTTATGTATCATACACATACG

We also prepared DNA template PMC1549 with T7 (AG) promoter underlined, EpCAM-CD3-hFc in bold. The structure of PMC1549 EPCAM-CD3-hFc with T7Ag promoter is shown below.

(SEQ ID NO: 23)
TAATACGACTCACTATAAGGAGAGAATACAAGCTTacatttgcttctgacacaactgtg
ttcactagcaacctcaaacagacaccATGGAGACAGACACACTCCTGCTATGGGTACTG
CTGCTCTGGGTTCCAGGTTCCACTGGCGCCGCTAGCCAGGTGCAACTCGTACAAAGCGG
TTCCGAACTGAAGAAACCTGGTGCGAGCGTAAAGGTGTCCTGTAAAGCATCTGGATAC
ACCTTCACCAACTACGGAATGAACTGGGTTCGACAGGCACCGGGCCAAGGCTT
GGAGTGGATGGGGTGGATCAACACCTACACGGGTGAACCTACGTACGCAGATG
ATTTCAAAGGTCGCTTCGTGTTTTCCCTGGATACTTCTGTGTCCACAGCGTACC
TCCAGATTTCCAGTCTGAAGGCCGAGGACACAGCCGTATATTACTGTGCTCGG
TGGTTGCGCGATTTTGATTATTGGGGCGCAGGTACTACTGTGACAGTAAGCTC
CGGCGGAGGCGGGTCAGGGGGCGGTGGATCCGGCGGAGGGGGATCAGAAATC
GTGCTCACTCAATCACCAGCGACCTTGAGCTTGTCACCTGGGGAGCGGGCGAC
GCTGTCCTGTAGTGCGTCATCATCTATTTCATATATGCACTGGTATCAACAGAA
GCCAGGACAGGCGCCTCGCCTGTTGATTTACGATACTAGCAAATTGGCAACGG
GGATACCCGCTCGATTCAGCGGCAGCGGATCTGGGACGGATTTCACATTGACC
ATTTCTAGCCTTGAACCAGAGGACTTCGCAGTATATTACTGCCATCAGAGGTCC
AGCTATCCATACACATTTGGCGGAGGGACGAAATTGGAAATCAAGGGGGGAGG
CGGGTCTGGGGGAGGCGGAAGTGGGGGAGGAGGAAGCGAAGTTCAGCTGCTC
GAATCCGGCGGCGGCCTTGTTCAGCCAGGTGGTAGCTTGAGGCTCAGTTGTGC
TGCATCTGGGTTTACATTCTCAACTTATGCGATGAACTGGGTGAGGCAAGCACC
TGGAAAGGGACTTGAGTGGGTCTCAAGAATTCGCTCCAAATACAACAACTATG
CGACGTATTACGCAGACTCAGTGAAAGGACGGTTTACGATATCACGGGACGAT
TCAAAGAATACACTGTATTTGCAGATGAATTCTCTTAGGGCCGAAGACACTGCC
GTATACTATTGTGTACGCCACGGTAATTTTGGCAATAGCTATGTATCTTGGTTC
GCGTACTGGGGCCAAGGCACCCTTGTTACTGTGTCTAGTGGGGGCGGGGGGA
GTGGTGGCGGAGGAAGTGGCGGGGGGGGATCTCAAGCGGTGGTTACTCAAGA
GCCCTCCCTTACTGTTTCTCCGGGCGGGACGGTCACCTTGACTTGTGGCAGTTC
AACAGGGGCAGTCACTACTAGTAATTATGCGAATTGGGTCCAAGAAAAGCCGG
GCCAAGCTTTCCGGGGACTCATCGGAGGAACAAATAAAAGGGCACCCGGCACA
CCCGCGCGCTTTTCCGGGAGTCTTCTGGGCGGCAAGGCAGCCCTCACTCTCTC
TGGGGCTCAACCTGAGGACGAGGCTGAGTACTATTGTGCCCTCTGGTACTCAA
ACCTGTGGGTCTTTGGAGGGGGAACCAAGCTTACGGTCTTGTCTAGAGAAAAC
CTGTATTTTCAGGGCACTCACACATGCCCACCGTGCCCAGCACCTGAACTCCTG
GGGGGACCGTCAGTCTTCCTCTTCCCCCCAAAACCCAAGGACACCCTCATGAT
CTCCCGGACCCCTGAGGTCACATGCGTGGTGGTGGACGTGAGCCACGAAGACC
CTGAGGTCAAGTTCAACTGGTACGTGGACGGCGTGGAGGTGCATAATGCCAAG
ACAAAGCCGCGGGAGGAGCAGTACAACAGCACGTACCGTGTGGTCAGCGTCCT
CACCGTCCTGCACCAGGACTGGCTGAATGGCAAGGAGTACAAGTGCAAGGTCT
CCAACAAAGCCCTCCCAGCCCCCATCGAGAAAACCATCTCCAAAGCCAAAGGG
CAGCCCCGAGAACCACAGGTGTACACCCTGCCCCCATCCCGGGATGAGCTGAC
CAAGAACCAGGTCAGCCTGACCTGCCTGGTCAAAGGCTTCTATCCCAGCGACA
TCGCCGTGGAGTGGGAGAGCAATGGGCAGCCGGAGAACAACTACAAGACCAC
GCCTCCCGTGCTGGACTCCGACGGCTCCTTCTTCCTCTACAGCAAGCTCACCGT
GGACAAGAGCAGGTGGCAGCAGGGGAACGTCTTCTCATGCTCCGTGATGCATG
AGGCTCTGCACAACCACTACACGCAGAAGAGCCTCTCCCTGTCTCCCGGGAAA
TGAGctcgctttcttgctgtccaatttctattaaaggttcctttgttccctaagtccaa
ctactaaactgggggatattatgaagggccttgagcatctggattctgcctaataaaaa
acatttattttcattgcagctcgctttcttgctgtccaatttctattaaaggttccttt
gttccctaagtccaactactaaactgggggatattatgaagggccttgagcatctgga
ttctgcctaataaaaaacatttattttcattgcaGTCGACTCTAGAAA
AAAAAAAAAAAAAAAAAAAAAAAAAAAAAAAAAAAAAAGGATCCCCGGGCGAGC
TCCCAAAAAAAAAAAAAAAAAAAAAAAAAAAAAACCGAATTCCTGCAGCTCGAGA
AAAAAAAAAAAAAAAAAAAAAAAAAAAAAAAAAAAAAAAAGATCTGGCGCGCCG
TAATCATGTCATAGCTGTTTCCTGTGTGAAATTGTTATCCGCTCACAATTCCACACAA
CATACGAGCCGGAAGCATAAAGTGTAAAGCCTGGGGTGCCTAATGAGTGAGCTAAC
TCACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGC
TCTTCCGCTTCCTCGCTCACTGACTCGCTGCGCTCGGTCGTTCGGCTGCGGCGAGCG
GTATCAGCTCACTCAAAGGCGGTAATACGGTTATCCACAGAATCAGGGGATAACGC
AGGAAAGAACATGTGAGCAAAAGGCCAGCAAAAGGCCAGGAACCGTAAAAAGGCC
GCGTTGCTGGCGTTTTTCCATAGGCTCCGCCCCCCTGACGAGCATCACAAAAATCGA
CGCTCAAGTCAGAGGTGGCGAAACCCGACAGGACTATAAAGATACCAGGCGTTTCC
CCCTGGAAGCTCCCTCGTGCGCTCTCCTGTTCCGACCCTGCCGCTTACCGGATACCTG
TCCGCCTTTCTCCCTTCGGGAAGCGTGGCGCTTTCTCATAGCTCACGCTGTAGGTATC
TCAGTTCGGTGTAGGTCGTTCGCTCCAAGCTGGGCTGTGTGCACGAACCCCCCGTTC
AGCCCGACCGCTGCGCCTTATCCGGTAACTATCGTCTTGAGTCCAACCCGGTAAGAC
ACGACTTATCGCCACTGGCAGCAGCCACTGGTAACAGGATTAGCAGAGCGAGGTAT
GTAGGCGGTGCTACAGAGTTCTTGAAGTGGTGGCCTAACTACGGCTACACTAGAAG
AACAGTATTTGGTATCTGCGCTCTGCTGAAGCCAGTTACCTTCGGAAAAAGAGTTGG
TAGCTCTTGATCCGGCAAACAAACCACCGCTGGTAGCGGTGGTTTTTTTGTTTGCAA
GCAGCAGATTACGCGCAGAAAAAAAGGATCTCAAGAAGATCCTTTGATCTTTTCTAC
GGGGTCTGACGCTCAGTGGAACGAAAACTCACGTTAAGGGATTTTGGTCATGAGATT
ATCAAAAAGGATCTTCACCTAGATCCTTTTAAATTAAAAATGAAGTTTTAAATCAAT
CTAAAGTATATATGAGTAAACTTGGTCTGACAGTTACCAATGCTTAATCAGTGAGGC
ACCTATCTCAGCGATCTGTCTATTTCGTTCATCCATAGTTGCCTGACTCCCCGTCGTG
TAGATAACTACGATACGGGAGGGCTTACCATCTGGCCCCAGTGCTGCAATGATACCG
CGAGACCCACGCTCACCGGCTCCAGATTTATCAGCAATAAACCAGCCAGCCGGAAG
GGCCGAGCGCAGAAGTGGTCCTGCAACTTTATCCGCCTCCATCCAGTCTATTAATTG
TTGCCGGGAAGCTAGAGTAAGTAGTTCGCCAGTTAATAGTTTGCGCAACGTTGTTGC
CATTGCTACAGGCATCGTGGTGTCACGCTCGTCGTTTGGTATGGCTTCATTCAGCTCC
GGTTCCCAACGATCAAGGCGAGTTACATGATCCCCCATGTTGTGCAAAAAAGCGGTT
AGCTCCTTCGGTCCTCCGATCGTTGTCAGAAGTAAGTTGGCCGCAGTGTTATCACTC
ATGGTTATGGCAGCACTGCATAATTCTCTTACTGTCATGCCATCCGTAAGATGCTTTT
CTGTGACTGGTGAGTACTCAACCAAGTCATTCTGAGAATAGTGTATGCGGCGACCGA
GTTGCTCTTGCCCGGCGTCAATACGGGATAATACCGCGCCACATAGCAGAACTTTAA
AAGTGCTCATCATTGGAAAACGTTCTTCGGGGCGAAAACTCTCAAGGATCTTACCGC
TGTTGAGATCCAGTTCGATGTAACCCACTCGTGCACCCAACTGATCTTCAGCATCTTT
TACTTTCACCAGCGTTTCTGGGTGAGCAAAAACAGGAAGGCAAAATGCCGCAAAAA
AGGGAATAAGGGCGACACGGAAATGTTGAATACTCATACTCTTCCTTTTTCAATATT
ATTGAAGCATTTATCAGGGTTATTGTCTCATGAGCGGATACATATTTGAATGTATTTA
GAAAAATAAACAAATAGGGGTTCCGCGCACATTTCCCCGAAAAGTGCCACCTGACG
TCTAAGAAACCATTATTATCATGACATTAACCTATAAAAATAGGCGTATCACGAGGC
CCTTTCGTCTCGCGCGTTTCGGTGATGACGGTGAAAACCTCTGACACATGCAGCTCC
CGGAGACGGTCACAGCTTGTCTGTAAGCGGATGCCGGGAGCAGACAAGCCCGTCAG
GGCGCGTCAGCGGGTGTTGGCGGGTGTCGGGGCTGGCTTAACTATGCGGCATCAGA
GCAGATTGTACTGAGAGTGCACCATTCGACGCTCTCCCTTATGCGACTCCTGCATTA
GGAAGCAGCCCAGTAGTAGGTTGAGGCCGTTGAGCACCGCCGCCGCAAGGAATGGT
GCATGCAAGGAGATGGCGCCCAACAGTCCCCCGGCCACGGGGCCTGCCACCATACC
CACGCCGAAACAAGCGCTCATGAGCCCGAAGTGGCGAGCCCGATCTTCCCCATCGG
TGATGTCGGCGATATAGGCGCCAGCAACCGCACCTGTGGCGCCGGTGATGCCGGCC
ACGATGCGTCCGGCGTAGAGGATCTGGCTAGCGATGACCCTGCTGATTGGTTCGCTG
ACCATTTCCGGGTGCGGGACGGCGTTACCAGAAACTCAGAAGGTTCGTCCAACCAA
ACCGACTCTGACGGCAGTTTACGAGAGAGATGATAGGGTCTGCTTCAGTAAGCCAG
ATGCTACACAATTAGGCTTGTACATATTGTCGTTAGAACGCGGCTACAATTAATACA
TAACCTTATGTATCATACACATACG

We also prepared EpCAM-CD3 antibody from the same nucleotide sequence as SEQ ID NO: 22, except with human Fc L234A, L235A and P329G mutations, called PMC1970 (SEQ ID NO: 24). Sequence of EPCAM-CD3 mutant Fc antibody is shown in bold. T7AG promoter underlined.

(SEQ ID NO: 24)
TAATACGACTCACTATAAGGAGAAAGCTTacatttgcttctgacacaactgtgttcact
agcaacctcaaacagacaccATGGAGACAGACACACTCCTGCTATGGGTACTGCTGCTC
TGGGTTCCAGGTTCCACTGGCGCCGCTAGCCAGGTGCAACTCGTACAAAGCGGTTCCGA
ACTGAAGAAACCTGGTGCGAGCGTAAAGGTGTCCTGTAAAGCATCTGGATACACCTTC
ACCAACTACGGAATGAACTGGGTTCGACAGGCACCGGGCCAAGGCTTGGAGTG
GATGGGGTGGATCAACACCTACACGGGTGAACCTACGTACGCAGATGATTTCA
AAGGTCGCTTCGTGTTTTCCCTGGATACTTCTGTGTCCACAGCGTACCTCCAGA
TTTCCAGTCTGAAGGCCGAGGACACAGCCGTATATTACTGTGCTCGGTGGTTG
CGCGATTTTGATTATTGGGGCGCAGGTACTACTGTGACAGTAAGCTCCGGCGG
AGGCGGGTCAGGGGGCGGTGGATCCGGCGGAGGGGGATCAGAAATCGTGCTC
ACTCAATCACCAGCGACCTTGAGCTTGTCACCTGGGGAGCGGGCGACGCTGTC
CTGTAGTGCGTCATCATCTATTTCATATATGCACTGGTATCAACAGAAGCCAGG
ACAGGCGCCTCGCCTGTTGATTTACGATACTAGCAAATTGGCAACGGGGATAC
CCGCTCGATTCAGCGGCAGCGGATCTGGGACGGATTTCACATTGACCATTTCT
AGCCTTGAACCAGAGGACTTCGCAGTATATTACTGCCATCAGAGGTCCAGCTAT
CCATACACATTTGGCGGAGGGACGAAATTGGAAATCAAGGGGGGAGGCGGGT
CTGGGGGAGGCGGAAGTGGGGGAGGAGGAAGCGAAGTTCAGCTGCTCGAATC
CGGCGGCGGCCTTGTTCAGCCAGGTGGTAGCTTGAGGCTCAGTTGTGCTGCAT
CTGGGTTTACATTCTCAACTTATGCGATGAACTGGGTGAGGCAAGCACCTGGA
AAGGGACTTGAGTGGGTCTCAAGAATTCGCTCCAAATACAACAACTATGCGAC
GTATTACGCAGACTCAGTGAAAGGACGGTTTACGATATCACGGGACGATTCAA
AGAATACACTGTATTTGCAGATGAATTCTCTTAGGGCCGAAGACACTGCCGTAT
ACTATTGTGTACGCCACGGTAATTTTGGCAATAGCTATGTATCTTGGTTCGCGT
ACTGGGGCCAAGGCACCCTTGTTACTGTGTCTAGTGGGGGGGGGGGGAGTGGT
GGCGGAGGAAGTGGCGGGGGGGGATCTCAAGCGGTGGTTACTCAAGAGCCCT
CCCTTACTGTTTCTCCGGGGGGACGGTCACCTTGACTTGTGGCAGTTCAACA
GGGGCAGTCACTACTAGTAATTATGCGAATTGGGTCCAAGAAAAGCCGGGCCA
AGCTTTCCGGGGACTCATCGGAGGAACAAATAAAAGGGCACCCGGCACACCCG
CGCGCTTTTCCGGGAGTCTTCTGGGCGGCAAGGCAGCCCTCACTCTCTCTGGG
GCTCAACCTGAGGACGAGGCTGAGTACTATTGTGCCCTCTGGTACTCAAACCT
GTGGGTCTTTGGAGGGGGAACCAAGCTTACGGTCTTGTCTAGAGAAAACCTGT
ATTTTCAGGGCACCCACACGTGCCCCCCTTGCCCAGCACCCGAAGCCGCAGGT
GGCCCATCAGTGTTTCTTTTTCCTCCAAAACCAAAAGACACACTCATGATCTCC
CGGACGCCTGAGGTGACCTGTGTAGTCGTAGACGTATCCCATGAGGACCCTGA
AGTAAAGTTTAACTGGTATGTAGACGGTGTGGAAGTACACAATGCCAAGACTA
AACCAAGAGAGGAACAGTATAACAGCACCTATAGGGTAGTTTCCGTGCTCACC
GTTCTCCACCAAGATTGGCTTAACGGTAAAGAATATAAATGTAAGGTGTCAAAT
AAGGCACTCGGAGCCCCGATCGAAAAGACCATCTCTAAAGCAAAAGGACAGCC
CAGGGAGCCACAAGTCTACACCCTGCCCCCATCCCGGGATGAGCTGACCAAGA
ACCAGGTCAGCCTGACCTGCCTGGTCAAAGGCTTCTATCCCAGCGACATCGCC
GTGGAGTGGGAGAGCAATGGGCAGCCGGAGAACAACTACAAGACCACGCCTC
CCGTGCTGGACTCCGACGGCTCCTTCTTCCTCTACAGCAAGCTCACCGTGGAC
AAGAGCAGGTGGCAGCAGGGGAACGTCTTCTCATGCTCCGTGATGCATGAGGC
TCTGCACAACCACTACACGCAGAAGAGCCTCTCCCTGTCTCCCGGGAAATAAGc
tcgctttcttgctgtccaatttctattaaaggttcctttgttccctaagtccaactact
aaactgggggatattatgaagggccttgagcatctggattctgcctaataaaaaacatt
tattttcattgcagctcgctttcttgctgtccaatttctattaaaggttcctttgttcc
ctaagtccaactactaaactgggggatattatgaagggccttgagcatctggattctgc
ctaataaaaaacatttattttcattgcaGTCGACTCTAGAAAAAAAAAAAAAAAAAAAA
AAAAAAAAAAAAAAAAAAAAAGGATCCCCGGGCGAGCTCCCA
AAAAAAAAAAAAAAAAAAAAAAAAAAAAACCGAATTCCTGCAGCTCGAGAAAAAA
AAAAAAAAAAAAAAAAAAAAAAAAAAAAAAAAAAAGATCTGGCGCGCCGTAATCA
TGTCATAGCTGTTTCCTGTGTGAAATTGTTATCCGCTCACAATTCCACACAACATACG
AGCCGGAAGCATAAAGTGTAAAGCCTGGGGTGCCTAATGAGTGAGCTAACTCACAT
TAATTGCGTTGCGCTCACTGCCCGCTTTCCAGTCGGGAAACCTGTCGTGCCAGCTGC
ATTAATGAATCGGCCAACGCGCGGGGAGAGGCGGTTTGCGTATTGGGCGCTCTTCCG
CTTCCTCGCTCACTGACTCGCTGCGCTCGGTCGTTCGGCTGCGGCGAGCGGTATCAG
CTCACTCAAAGGCGGTAATACGGTTATCCACAGAATCAGGGGATAACGCAGGAAAG
AACATGTGAGCAAAAGGCCAGCAAAAGGCCAGGAACCGTAAAAAGGCCGCGTTGC
TGGCGTTTTTCCATAGGCTCCGCCCCCCTGACGAGCATCACAAAAATCGACGCTCAA
GTCAGAGGTGGCGAAACCCGACAGGACTATAAAGATACCAGGCGTTTCCCCCTGGA
AGCTCCCTCGTGCGCTCTCCTGTTCCGACCCTGCCGCTTACCGGATACCTGTCCGCCT
TTCTCCCTTCGGGAAGCGTGGCGCTTTCTCATAGCTCACGCTGTAGGTATCTCAGTTC
GGTGTAGGTCGTTCGCTCCAAGCTGGGCTGTGTGCACGAACCCCCCGTTCAGCCCGA
CCGCTGCGCCTTATCCGGTAACTATCGTCTTGAGTCCAACCCGGTAAGACACGACTT
ATCGCCACTGGCAGCAGCCACTGGTAACAGGATTAGCAGAGCGAGGTATGTAGGCG
GTGCTACAGAGTTCTTGAAGTGGTGGCCTAACTACGGCTACACTAGAAGAACAGTAT
TTGGTATCTGCGCTCTGCTGAAGCCAGTTACCTTCGGAAAAAGAGTTGGTAGCTCTT
GATCCGGCAAACAAACCACCGCTGGTAGCGGTGGTTTTTTTGTTTGCAAGCAGCAGA
TTACGCGCAGAAAAAAAGGATCTCAAGAAGATCCTTTGATCTTTTCTACGGGGTCTG
ACGCTCAGTGGAACGAAAACTCACGTTAAGGGATTTTGGTCATGAGATTATCAAAA
AGGATCTTCACCTAGATCCTTTTAAATTAAAAATGAAGTTTTAAATCAATCTAAAGT
ATATATGAGTAAACTTGGTCTGACAGTTACCAATGCTTAATCAGTGAGGCACCTATC
TCAGCGATCTGTCTATTTCGTTCATCCATAGTTGCCTGACTCCCCGTCGTGTAGATAA
CTACGATACGGGAGGGCTTACCATCTGGCCCCAGTGCTGCAATGATACCGCGAGAC
CCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCG
GGAAGCTAGAGTAAGTAGTTCGCCAGTTAATAGTTTGCGCAACGTTGTTGCCATTGC
TACAGGCATCGTGGTGTCACGCTCGTCGTTTGGTATGGCTTCATTCAGCTCCGGTTCC
CAACGATCAAGGCGAGTTACATGATCCCCCATGTTGTGCAAAAAAGCGGTTAGCTCC
TTCGGTCCTCCGATCGTTGTCAGAAGTAAGTTGGCCGCAGTGTTATCACTCATGGTT
ATGGCAGCACTGCATAATTCTCTTACTGTCATGCCATCCGTAAGATGCTTTTCTGTGA
CTGGTGAGTACTCAACCAAGTCATTCTGAGAATAGTGTATGCGGCGACCGAGTTGCT
CTTGCCCGGCGTCAATACGGGATAATACCGCGCCACATAGCAGAACTTTAAAAGTG
CTCATCATTGGAAAACGTTCTTCGGGGCGAAAACTCTCAAGGATCTTACCGCTGTTG
AGATCCAGTTCGATGTAACCCACTCGTGCACCCAACTGATCTTCAGCATCTTTTACTT
TCACCAGCGTTTCTGGGTGAGCAAAAACAGGAAGGCAAAATGCCGCAAAAAAGGG
AATAAGGGCGACACGGAAATGTTGAATACTCATACTCTTCCTTTTTCAATATTATTG
AAGCATTTATCAGGGTTATTGTCTCATGAGCGGATACATATTTGAATGTATTTAGAA
AAATAAACAAATAGGGGTTCCGCGCACATTTCCCCGAAAAGTGCCACCTGACGTCT
AAGAAACCATTATTATCATGACATTAACCTATAAAAATAGGCGTATCACGAGGCCCT
TTCGTCTCGCGCGTTTCGGTGATGACGGTGAAAACCTCTGACACATGCAGCTCCCGG
AGACGGTCACAGCTTGTCTGTAAGCGGATGCCGGGAGCAGACAAGCCCGTCAGGGC
GCGTCAGCGGGTGTTGGCGGGTGTCGGGGCTGGCTTAACTATGCGGCATCAGAGCA
GATTGTACTGAGAGTGCACCATTCGACGCTCTCCCTTATGCGACTCCTGCATTAGGA
AGCAGCCCAGTAGTAGGTTGAGGCCGTTGAGCACCGCCGCCGCAAGGAATGGTGCA
TGCAAGGAGATGGCGCCCAACAGTCCCCCGGCCACGGGGCCTGCCACCATACCCAC
GCCGAAACAAGCGCTCATGAGCCCGAAGTGGCGAGCCCGATCTTCCCCATCGGTGA
TGTCGGCGATATAGGCGCCAGCAACCGCACCTGTGGCGCCGGTGATGCCGGCCACG
ATGCGTCCGGCGTAGAGGATCTGGCTAGCGATGACCCTGCTGATTGGTTCGCTGACC
ATTTCCGGGTGCGGGACGGCGTTACCAGAAACTCAGAAGGTTCGTCCAACCAAACC
GACTCTGACGGCAGTTTACGAGAGAGATGATAGGGTCTGCTTCAGTAAGCCAGATG
CTACACAATTAGGCTTGTACATATTGTCGTTAGAACGCGGCTACAATTAATACATAA
CCTTATGTATCATACACATACG

The translated antibody sequence is shown below with mutations (L234A, L235A and P329G) underlined and in bold.

(SEQ ID NO: 25)
METDTLLLWVLLLWVPGSTGAASQVQLVQSGSELKKPGASVKVSCKASGY
TFTNYGMNWVRQAPGQGLEWMGWINTYTGEPTYADDFKGRFVFSLDTSVS
TAYLQISSLKAEDTAVYYCARWLRDFDYWGAGTTVTVSSGGGGSGGGGSG
GGGSEIVLTQSPATLSLSPGERATLSCSASSSISYMHWYQQKPGQAPRLL
IYDTSKLATGIPARFSGSGSGTDFTLTISSLEPEDFAVYYCHQRSSYPYT
FGGGTKLEIKGGGGSGGGGSGGGGSEVQLLESGGGLVQPGGSLRLSCAAS
GFTFSTYAMNWVRQAPGKGLEWVSRIRSKYNNYATYYADSVKGRFTISRD
DSKNTLYLQMNSLRAEDTAVYYCVRHGNFGNSYVSWFAYWGQGTLVTVSS
GGGGSGGGGSGGGGSQAVVTQEPSLTVSPGGTVTLTCGSSTGAVTTSNYA
NWVQEKPGQAFRGLIGGTNKRAPGTPARFSGSLLGGKAALTLSGAQPEDE
AEYYCALWYSNLWVFGGGTKLTVLSRENLYFQGTHTCPPCPAPEAAGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALGAPIEKTISKA
KGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQK
SLSLSPGK

The sequence of CH2-CH3 of human IgG1 Fc with L234A, L235A, and P329G substitutions is shown below.

(SEQ ID NO: 26)
APEAAGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALGA
PIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHE
ALHNHYTQKSLSLSPGK

### Example 6. Expression of EPCAM-CD3 Antibodies (FIG. 1A)

293S cells were grown in Freestyle F17 Expression serum free medium with 8 mM L-Glutamine (or GlutaMAX); 0.1% Pluoronic F-68. For transfection, NanoFect Transfection Reagent was used at ratio 3:1 (3 microliters for 1 microg DNA). The supernatant was harvested after 3-7 days of transfection.

The antibody protein supernatants were expressed and run on the SDS gel at reduced and non-reduced condition (adding beta-mercapto-ethanol to lysis buffer) (FIG. 2). The gel at reduced conditions showed 4 bands. The protein was also purified using protein A or G columns. The purification was done with Millipore Sigma Protein A beads and Thermo IgG Elution buffer (Catalog number: 21004). In SDS gel, the purified EPCAM antibody showed around 190 kDA band at non-reducing conditions.

### Example 7. Binding of CD3 and EPCAM Antigens by FACS (FIG. 1A Structure)

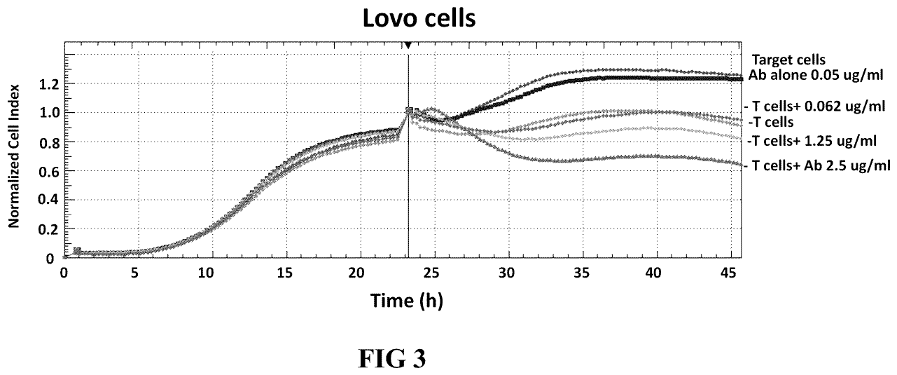

The FACS using bispecific EPCAM-CD3 antibodies (FIG. 1A) demonstrates that antibodies bind to EPCAM in EPCAM-positive cells, and CD3 using T cells (FIG. 3).

The bispecific antibodies were tested with EPCAM-positive and EPCAM-negative cell lines. CD3-positive T cells were used for testing binding to CD3. Bispecific antibodies had positive binding with both EPCAM and CD3 antigens. By FACS, EPCAM-CD3 antibody bound to EPCAM-positive Lovo cell line, and did not bind to EPCAM-negative HL-60 cell line. Also, positive staining of EPCAL-CD3 antibody was shown on CD3-positive T cells.

### Example 8. Cytotoxic Activity of EPCAM-CD3 Antibody With T Cells on EPCAM-Positive Target Cell Line (FIG. 1A Structure)

Antibody supernatants together with T cells were used for RTCA assay. EPCAM-CD3 antibody plus T cells killed EPCAM-positive cells (FIG. 3). T cells alone also did not kill target cells (FIG. 3). This demonstrates high specificity of the bispecific antibody when it is used together with T cells. The results confirm mechanism of bringing T cells to cancer cells through bispecific antibody by binding to CD3 antigen in T cells. FIG. 3 also showed that the bispecific antibodies had dose-dependent activity.

### Example 9. Bivalent Humanized CROSSFAB EPCAM-CD3 SCFV Plus T Cells Specifically Secreted IFN-Gamma (FIG. 1A Structure)

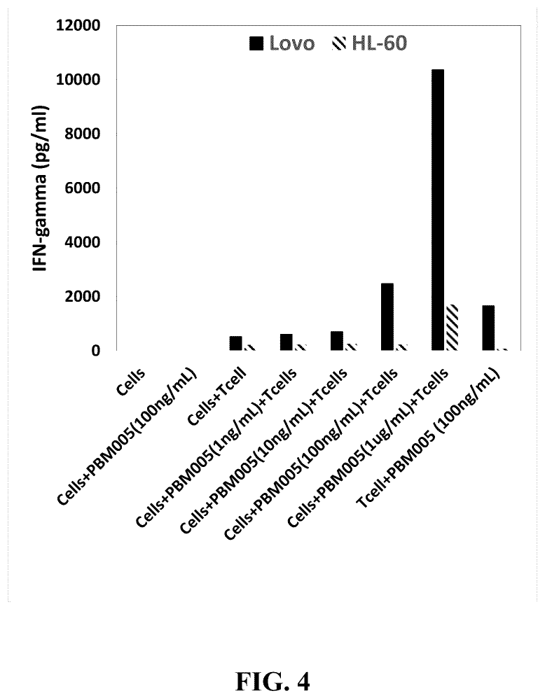

The bivalent, bispecific humanized EPCAM CrossFAB with CD3 ScFv antibody (shown FIG. 1A, PBM005) induced IFN-gamma secretion by T cells against EPCAM-positive Lovo cells. There was no induction of IFN-gamma secretion by T cells against EPCAM-negative HL-60 cells (FIG. 4). The results demonstrate high and specific activity of this antibody.

### Example 10. EPCAM SCFV-CD3 SCFV Antibody With T Cells Specifically Killed EPCAM-Positive Cells and Secreted IFN-Gamma (FIG. 1B Structure)

EpCAM Scfv-CD3 ScFv-His tag (PBM0065) was purified using Ni-NTA purification column and run on SDS gel. The results showed one band with molecular weight about 55 kDa.

By FACS, EpCAM Scfv-CD3 ScFv (PBM0065) showed binding to Lovo cells, but not to HL-60 cells.

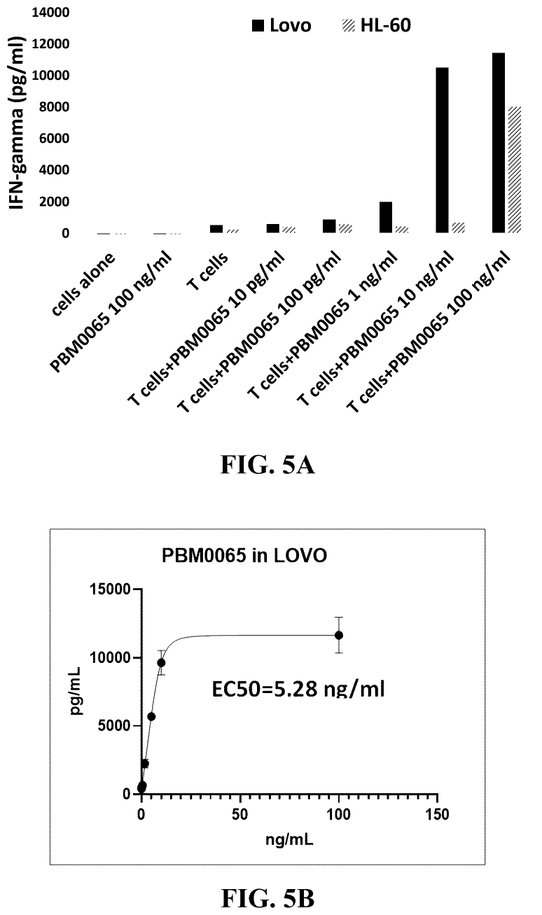

EpCAM Scfv-CD3 ScFv (PBM0065) with T cells caused secretion of significant level of IFN-gamma with EPCAM-positive Lovo cells, but not with EPCAM-negative HL-60 cells (FIG. 5A). The EC50 based on IFN-gamma secretion was shown as 5.28 ng/ml (FIG. 5B).

### Example 11. EPCAM SCFV-CD3 SCFV-Human FC Antibody (PBM0070) With T Cells Specifically Killed EPCAM-Positive Cells and Secreted IFN-Gamma (FIG. 1C Structure)

The bispecific univalent humanized EPCAM ScFv-CD3 Scfv-human Fc antibody with structure as shown in FIG. 1C was run as one band on SDS gel at non-reduced conditions with molecular weight equal to 162 kDa and equal to 81 kDa at reduced conditions corresponding to monomeric protein versus dimeric at non-reduced conditions.

Humanized EPCAM ScFv-CD3 ScFv antibody bound to EPCAM in EPCAM-positive Lovo cells, but not EPCAM-negative HL-60 cells. The antibody also bound to CD3 by FACS analysis.

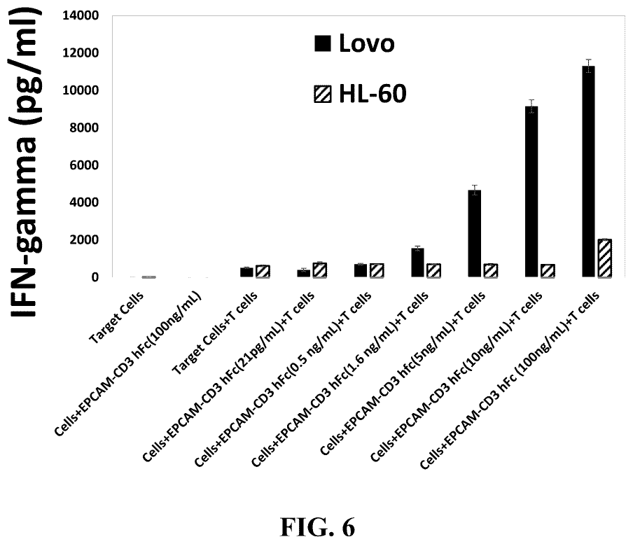

The EPCAM-CD3 Ab with T cells secreted significant level of IFN-gamma with EPCAM-positive cells but not with EPCAM-negative cells HL-60 (FIG. 6). EC50 of IFN-gamma secretion EPCAM-CD3 antibody with T cells was 6.1 ng/ml (FIG. 7)

### Example 12. EPCAM SCFV-CD3 SCFV-Human FC (PMC1396) Produced by RNA Transcription Shows Good Efficacy In Vitro (FIG. 1C Structure)

The EPCAM ScFV-CD3 ScFv-human Fc (PBM0070) antibody structure (FIG. 1C) was used as a template for RNA transcription. The RNA was generated using in vitro transcription kit. This RNA was transfected to 293 cells and supernatant was collected at 24 and 48 hours to run on SDS gel. The 81 kDa band was present at reduced conditions at 24 and 48 hours after transfection with PCM1396 EPCAM Scfv-CD3 Scfv-hFc RNA. The results show bispecific antibody was successfully produced by RNA transcription in vitro.

By FACS, the EPCAM-CD3-hFc protein collected in supernatant 48 hours after RNA transfection bound to EPCAM-positive Lovo cells and CD3-positive T cells, but not to EPCAM-negative HL-60 cells. We also tested by FACS two known EPCAM-negative cell lines (COLO320 and COLO741) and confirmed antibody specificity.

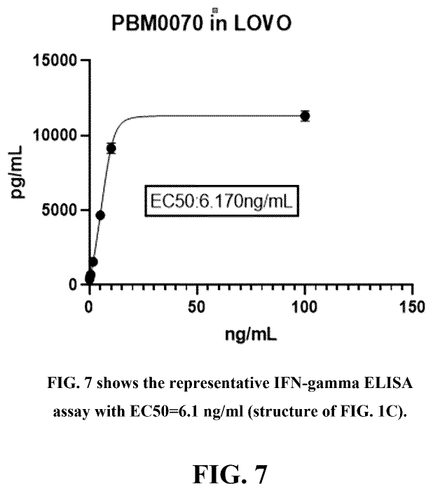

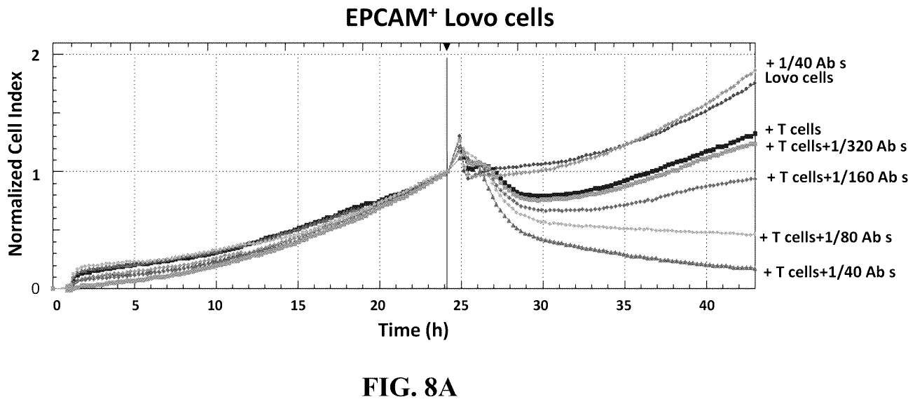

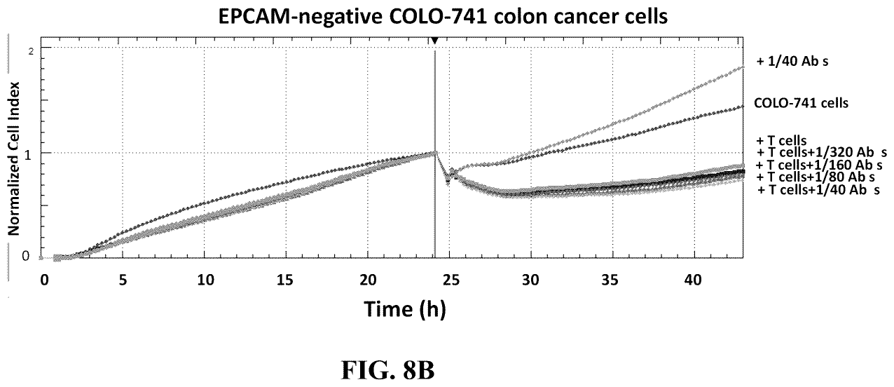

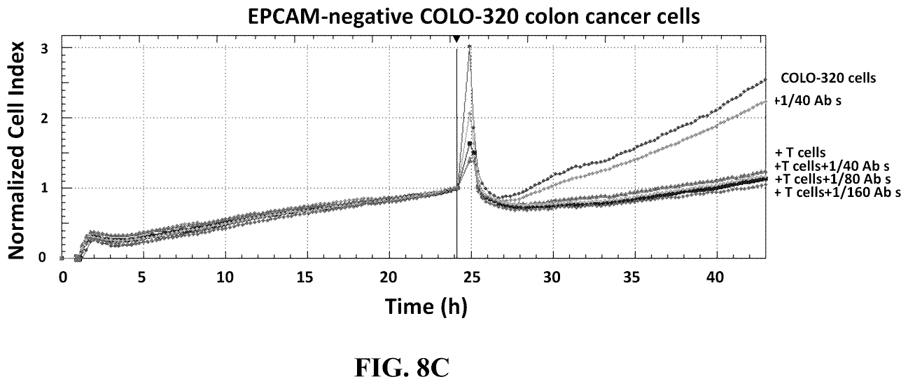

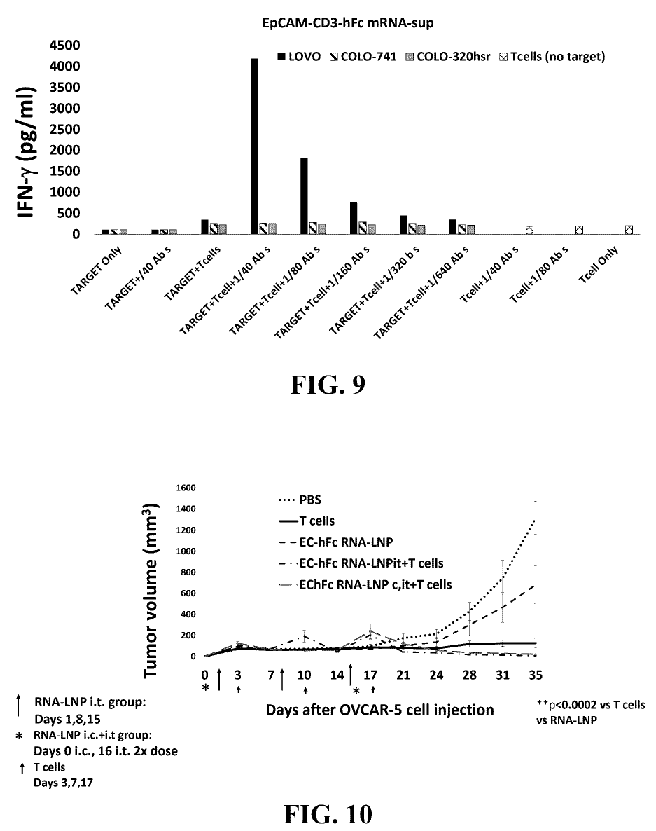

The EPCAM-CD3 antibody killed EPCAM positive Lovo cells but not EPCAM-negative COLO741 or COLO320 cells by RTC assay (FIG. 8). The antibody secreted IFN-gamma with T cells in a dose dependent manner with Lovo cells but not with COLO741 and COLO320 cells (FIG. 9).

### Example 13. Materials for Preparation of LNP

SM-102: 8-[(2-hydroxyethyl)[6-oxo-6-(undecyloxy)hexyl]amino]-octanoic acid, 1-octylnonyl ester; CAS number: 2089251-47-6
ALC-0315: 2-hexyl-decanoic acid, 1,1′-[[(4-hydroxybutyl)imino]di-6,1-hexanediyl] ester
CAS number 2036272-55-4.
ALC-0159: α-[2-(ditetradecylamino)-2-oxoethyl]-ω-methoxy-poly(oxy-1,2-ethanediyl)
CAS: 1849616-42-7
DMG-PEG2000: 1,2-dimyristoyl-rac-glycero-3-methoxypolyethylene glycol-2000
DSPC: Distearoylphosphatidylcholine
Cholesterol
Nanoparticles can be prepared either with SM-102 or ALC315 ionizing lipid.

### Example 14. In Vitro Transcription and MRNA Encapsulation to LNP (FIG. 1C Structure)

The mRNA was in vitro transcribed from a DNA template using the HiScribe T7 mRNA Kit with CleanCap Reagent AG (NEB #E2080). In brief, a DNA template, 0.5×T7 CleanCap Reagent AG Reaction Buffer, 5 mM of ATP, CTP, pseudo-UTP, and GTP were added to 4 mM of CleanCapAG and T7 polymerase mix for 2 h at 37 C. Then, DNAse I treatment was performed for 15 min at 37° C. The mRNA was purified with the Monarch RNA Cleanup Kit (T2050) according to the manufacturer's protocol.

To generate an mRNA-LNP complex, an aqueous solution of mRNA in 100 mM sodium acetate (pH 4.0) was combined with a lipid mix containing the ethanol phase of SM-102 (Cayman), DSPC (Avanti), cholesterol (Sigma), and DMG-PEG2000 (Cayman) (at a molar % ratio of 50:10:38.5:1.5, respectively) at a flow rate ratio of 3:1 (aqueous:organic) using the PreciGenome Flex S System (San Jose, CA, USA). The mRNA-LNPs were purified and concentrated using Amicon® Ultra-15 centrifugal filter units (30-100 kDa). The size, zeta-potential, and polydispersity index (PDI) of the mRNA-LNPs were detected using an Anton Paar Litesizer 500 System, and the encapsulation efficiency was checked with the Quant-it™ RiboGreen RNA assay Kit. The mean mRNA-LNP size was 104 nm; the PDI was 0.14; and the encapsulation efficiency was 91.8%.

### Example 15. Expression of EPCAM-CD3-HFC Antibody in Cancer Cell Lines Transfected With EPCAM-CD3-LNP (FIG. 1C Structure)

We transfected 1 μg of EPCAM-CD3 mRNA-LNP (Example 14) to 0.5-1×10⁶ cells of different ovarian SKOV-3, OVCAR-3 and colorectal OVCAR-5 cell lines. We found that EpCAM-CD3 was secreted from OVCAR-5 cancer cell line. The supernatant bound EpCAM-positive Lovo cells and did not bind EpCAM-negative cells. The results showed that the antibody was generated inside cancer cell lines.

Example 16. In Vivo Efficacy of EPCAM-CD3-HFC-LNP With T Cells In Vivo in Mice (FIG. 1C Structure)

To test that EPCAM-CD3 antibodies could be generated from EPCAM-CD3 RNA-LNP in vivo, we used EpCAM-CD3-hFc RNA-LNP (Example 14) and injected it into mice either alone or together with T cells using OVCAR-5 xenograft tumor model. We injected 4×10⁶ subcutaneously colorectal OVCAR-5 cell line into NSG mice and then injected EpCAM-CD3-hFc-RNA-LNP on days 1, 8, and 15 into tumor. Human T cells were delivered intravenously into mice two days after EpCAM-CD3-hFc-RNA-LNP (on days 3, 10, 17). EPCAM-CD3 RNA-LNP injected intro tumors with T cells significantly decreased OVCAR-5 tumor xenograft growth versus T cells alone or EpCAM-CD3-hFc RNA-LNP alone (FIG. 10).

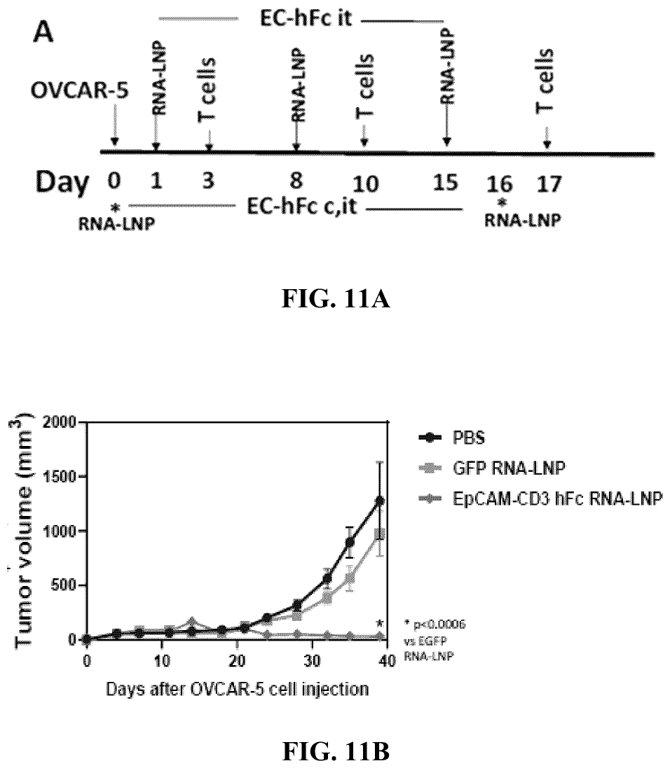

In another experiment, FIG. 10 shows significant decrease of tumors with 3 intratumoral injections of EpCAM-CD3 RNA-LNP and only one injection of T cells using PVCAR-5 xenograft mouse model. EpCAM-CD3 hFc mRNA-LNPs was delivered intratumorally on days 6, 13, 20 and 25 with only a single injection of T cells by i.v. on day 8 (FIG. 11A). As a negative control, GFP mRNA-LNPs was used. EpCAM-CD3 mRNA-LNPs significantly blocked OVCAR-5 xenograft tumor growth while GFP mRNA-LNPs did not (FIG. 11B). The images of tumors at the end of treatment were determined. There was a significant reduction of tumor size and weight in the EpCAM-CD3 hFc mRNA-LNP and T cell treated group versus the PBS and GFP mRNA-LNP-treated group. In addition, serum from treated mice was collected and there was no increase of blood toxicology markers (AST, ALT, Amylase and LDH) by EpCAM-CD3 hFc mRNA-LNP treatment versus GFP mRNA-LNP treatment suggesting no toxicity of EpCAM-CD3 mRNA-LNPs. Thus, Intratumoral delivery of the EpCAM-CD3 human Fc mRNA-LNPs into OVCAR-5 tumor xenografts with intravenous injection of T cells significantly decreased xenograft tumor growth.

This result shows that bispecific antibodies can be generated in vivo, which significantly decreases cost of antibody manufacturing. Bispecific EPCAM-CD3 antibodies produced after delivery of RNA-LNP and with T cells decreased mouse xenograft tumor growth.

### REFERENCES

1. U. Schnell, V. Cirulli and B. N. Giepmans: EpCAM: structure and function in health and disease. Biochim Biophys Acta, 1828(8), 1989-2001 (2013) doi:10.1016/j.bbamem.2013.04.018
2. J. Ni, P. J. Cozzi, W. Duan, S. Shigdar, P. H. Graham, K. H. John and Y. Li: Role of the EpCAM (CD326) in prostate cancer metastasis and progression. Cancer Metastasis Rev, 31(3-4), 779-91 (2012) doi:10.1007/s10555-012-9389-1
3. L. Huang, Y. Yang, F. Yang, S. Liu, Z. Zhu, Z. Lei and J. Guo: Functions of EpCAM in physiological processes and diseases (Review). Int J Mol Med, 42(4), 1771-1785 (2018) doi:10.3892/ijmm.2018.3764
4. E. Bremer and W. Helfrich: EpCAM-targeted induction of apoptosis. Front Biosci, 13, 5042-9 (2008) doi:10.2741/3062
5. R. Berahovich, H. Zhou, S. Xu, Y. Wei, J. Guan, J. Guan, H. Harto, S. Fu, K. Yang, S. Zhu, L. Li, L. Wu and V. Golubovskaya: CAR-T Cells Based on Novel BCMA Monoclonal Antibody Block Multiple Myeloma Cell Growth. Cancers (Basel), 10(9) (2018) doi:10.3390/cancers10090323
6. M. Bacac, T. Fauti, J. Sam, S. Colombetti, T. Weinzierl, D. Ouaret, W. Bodmer, S. Lehmann, T. Hofer, R. J. Hosse, E. Moessner, O. Ast, P. Bruenker, S. Grau-Richards, T. Schaller, A. Seidl, C. Gerdes, M. Perro, V. Nicolini, N. Steinhoff, S. Dudal, S. Neumann, T. von Hirschheydt, C. Jaeger, J. Saro, V. Karanikas, C. Klein and P. Umana: A Novel Carcinoembryonic Antigen T-Cell Bispecific Antibody (CEA TCB) for the Treatment of Solid Tumors. Clin Cancer Res, 22(13), 3286-97 (2016) doi:10.1158/1078-0432.CCR-15-1696
7. Wong, N. A.; Warren, B. F.; Piris, J.; Maynard, N.; Marshall, R.; Bodmer, W. F. EpCAM and gpA33 are markers of Barrett's metaplasia.
J. Clin. Pathol. 2006, 59, 260-263
8. Almagro, J. C.; Fransson, J. Humanization of antibodies. Front. Biosci. 2008, 13, 1619-1633.
9. Golubovskaya, V.; Berahovich, R.; Zhou, H.; Xu, S.; Harto, H.; Li, L.; Chao, C. C.; Mao, M. M.; Wu, L. CD47-CAR-T Cells Effectively Kill Target Cancer Cells and Block Pancreatic Tumor Growth. Cancers 2017, 9, 139.
10. Bacac, M.; Fauti, T.; Sam, J.; Colombetti, S.; Weinzierl, T.; Ouaret, D.; Bodmer, W.; Lehmann, S.; Hofer, T.; Hosse, R. J.; et al. A Novel Carcinoembryonic Antigen T-Cell Bispecific Antibody (CEA TCB) for the Treatment of Solid Tumors. Clin. Cancer Res. 2016, 22, 3286-3297.
11. Reinhard, K.; Rengstl, B.; Oehm, P.; Michel, K.; Billmeier, A.; Hayduk, N.; Klein, O.; Kuna, K.; Ouchan, Y.; Woll, S.; et al. An RNA vaccine drives expansion and efficacy of claudin-CAR-T cells against solid tumors. Science 2020, 367, 446-453

What is claimed is:

1. A bispecific antigen-binding molecule comprising humanized EPCAM VH, a first linker, humanized EPCAM VL, a second linker, CD3 VH, a third linker, CD3 VL, and a human Fc domain.

2. The bispecific antigen-binding molecule of claim 1, wherein the EPCAM VH has the amino acid sequence of SEQ ID NO: 10, the EPCAM VL has the amino acid sequence of SEQ ID NO: 4, the CD3 VH has the amino acid sequence of SEQ ID NO: 11, and the CD3 VL has the amino acid sequence of SEQ ID NO: 7.

3. The bispecific antigen-binding molecule of claim 1, wherein the human Fc domain comprises a hinge linker, and CH2-CH3 of human IgG1, optionally substituted with one or two amino acid substitutions.

4. The bispecific antigen-binding molecule of claim 2, wherein the human Fc domain comprises a hinge linker, and CH2-CH3 of human IgG1, optionally substituted with one or two amino acid substitutions.

5. The bispecific antigen-binding molecule of claim 2, wherein the Fc domain comprises one or more amino acid substitutions selected from the group of L234A, L235A, and P329G (EU numbering).

6. The bispecific antigen-binding molecule of claim 2, wherein each first, second, and third link has the amino acid sequence of (GGGGS)n, and n=1-5.

7. The bispecific antigen-binding molecule of claim 1, having the amino acid sequence of SEQ ID NO: 19.

8. The bispecific antigen-binding molecule of claim 1, wherein the human Fc domain comprises the amino acid sequence of SEQ ID NO: 20 or 26.

9. The bispecific antigen-binding molecule of claim 1, comprising EPCAM VH having the amino acid sequence of SEQ ID NO: 10, a first linker, EPCAM VL having the amino acid sequence of SEQ ID NO: 4, a second linker, CD3 VH having the amino acid sequence of SEQ ID NO: 11, a third linker, CD3 VL having the amino acid sequence of SEQ ID NO: 7, wherein the first, the second, and the third linkers are the same or different.

10. The bispecific antigen-binding molecule of claim 9, wherein each link has the amino acid sequence of (GGGGS)₃.

11. An isolated DNA sequence comprising (a) a promoter coding sequence, (b) 5′-UTR (untranslated region) coding sequence, (c) a coding sequence to encode the bispecific antigen-binding molecule of claim 1, (d) a 3′-UTR coding sequence, and (e) a poly A tail sequence.

12. A method for producing the bispecific antigen-binding molecule in cells, comprising the steps of:
obtaining the DNA sequence of claim 11,
transcribing the DNA sequence to mRNA with RNA polymerase in vitro,
mixing the mRNA with lipid nanoparticles (LNP) to form mRNA-LNP complex,
transfecting the mRNA-encapsulated LNPs into cells, and
translating the mRNA in the cells to produce the bispecific antigen-binding molecule in cells.

13. The method of claim 12, wherein the cells are cancer cells.

14. A method for treating cancer, comprising the steps of:
obtaining the DNA sequence of claim 11,
transcribing the DNA sequence to mRNA with RNA polymerase in vitro,
mixing the mRNA with lipid nanoparticles (LNP) to form mRNA-LNP complex, and
injecting the mRNA-lipid nanoparticle complex into tumors.

15. The method of claim 14, further comprising injecting T cells intravenously.

16. A bispecific antigen-binding molecule comprising: (a) a first and a second antigen-binding moiety each of which is a humanized Fab molecule capable of specific binding to human EPCAM, and each comprises a heavy chain variable region (EPCAM VH) having the amino acid sequence of SEQ ID NO: 10 and a light chain variable region (EPCAM VL) having the amino acid sequence of SEQ ID NO: 4; (b) a third antigen-binding moiety which is a Fab molecule capable of specific binding to human CD3 epsilon (CD3), the third antigen-binding moiety comprises a heavy chain variable region (CD3 VH) having the amino acid sequence of SEQ ID NO: 11 and a light chain variable region (CD3 VL) having the amino acid sequence of SEQ ID NO: 7, wherein the third antigen-binding moiety is a crossover Fab molecule, in which the constant regions of the Fab light chain and the Fab heavy chain are exchanged; and (c) an human Fc domain comprising a first subunit and a second subunit capable of stable association; wherein the Fab heavy chain of the third antigen-binding moiety is (i) fused at the N-terminus to the C-terminus of the Fab heavy chain of the first antigen-binding moiety (CH1), and (ii) fused at the C-terminus to the N-terminus of the first subunit of the Fc knob domain, and wherein the second antigen-binding moiety is fused at the C-terminus of the Fab heavy chain (CH1) to the N-terminus of the second subunit of the Fc hole domain.

17. The bispecific antigen-binding molecule of claim 16, wherein the human Fc domain comprises one or more amino acid substitutions promoting the association of the first and the second subunit of the Fc domain.

18. A bispecific antigen-binding molecule comprising two binding moieties to EPCAM, and one binding moiety to CD3 epsilon, the molecule comprises the amino acid sequences of SEQ ID NO: 5, 8, 12, and 14, in a molar ratio of 2:1:1:1.
